(19) United States
(12) Patent Application Publication
(10) Pub. No.: US 2023/0190134 A1
(43) Pub. Date: Jun. 22, 2023

(54) SIZER FOR MEASURING AN ANATOMICAL STRUCTURE, WITH DIGITAL MONITORING, DISPLAY, RECORDING, AND COMMUNICATING SYSTEM

(71) Applicant: Cilag GmbH International, Zug (CH)

(72) Inventors: Frederick E. Shelton, IV, Hillsboro, OH (US); Kevin M. Fiebig, Cincinnati, OH (US); Glen W. Ellison, Maineville, OH (US); Tylor C. Muhlenkamp, Cincinnati, OH (US)

(21) Appl. No.: 17/552,496

(22) Filed: Dec. 16, 2021

(51) Int. Cl. A61B 5/107; G06T 7/00; A61B 1/018; A61B 5/06; A61F 2/04

(52) U.S. Cl. CPC A61B 5/1076; G06T 7/0012; A61B 1/018; A61B 5/064; A61F 2/04; G06T 2207/10068; A61F 2002/044

(57) ABSTRACT

An apparatus includes a body, a shaft assembly, and a first sensor. The shaft assembly extends distally from the body. The shaft assembly includes a first outer shaft member and a second outer shaft member. The first outer shaft member has a first distal end. The second outer shaft member has a second distal end. The second outer shaft member is translatable relative to the first outer shaft member. A distal portion of the second outer shaft member is configured to encircle an anatomical structure of a patient and couple the second distal end with the first distal end while encircling the anatomical structure of the patient. A first sensor is configured to detect an outer diameter of the anatomical structure encircled by the distal portion of the second outer shaft member.

### BACKGROUND

In some instances, it may be desirable to place a medical implant within or surrounding a biological lumen/passageway in order to improve or assist the function of, or otherwise affect, the biological lumen/passageway. Examples of such biological lumens/passageways include, but are not limited to, the esophagus, a fallopian tube, a urethra, or a blood vessel. Some biological passages normally function by expanding and contracting actively or passively to regulate the flow of solids, liquids, gasses, or a combination thereof. The ability of a biological passage to expand and contract may be compromised by defects or disease. One merely illustrative example of a condition associated with decreased functionality of a body passage is Gastro Esophageal Reflux Disease (“GERD”), which effects the esophagus.

A normal, healthy, esophagus is a muscular tube that carries food from the mouth, through the chest cavity and into the upper part of the stomach. A small-valved opening in the esophagus, called the lower esophageal sphincter (“LES”), regulates the passage of food from the esophagus into the stomach, as well as the passage of acidic fluids and food from the stomach toward the esophagus. The LES may also regulate stomach intra-gastric pressures. A healthy LES may contain pressure of gasses within the stomach at around 10 mm Hg greater than normal intragastrical pressure, thereby impeding acidic gases/fluids from refluxing from the stomach back into the esophagus. When functioning properly, a pressure difference greater than 10 mm Hg may regulate when the LES opens to allow gasses to be vented from the stomach toward the esophagus.

If the LES relaxes, atrophies, or degrades for any reason, the LES may cease functioning properly. Therefore, the LES may fail to sufficiently contain pressure of gasses within the stomach such that acidic contents of the stomach may travel back into the esophagus, resulting in reflux symptoms. Two primary components that control the LES are the intrinsic smooth muscle of the distal esophagus wall and the skeletal muscle of the crural diaphragm or esophageal hiatus. A causation of esophageal reflux, which may be associated with GERD, is relaxation of one or both of the smooth muscle of the distal esophagus wall or the hiatal diaphragm sphincter mechanisms. Chronic or excessive acid reflux exposure may cause esophageal damage. Conventionally, treatment for GERD may involve either open or endoscopic surgical procedures. Some procedures may include a fundoplication that mobilizes the stomach relative to the lower esophagus; or suturing a pleat of tissue between the LES and the stomach to make the lower esophagus tighter.

Examples of devices and methods that have been developed to treat anatomical lumens by providing sphincter augmentation are described in U.S. Pat. No. 7,175,589, entitled “Methods and Devices for Luminal and Sphincter Augmentation,” issued Feb. 13, 2007, the disclosure of which is incorporated by reference herein, in its entirety; U.S. Pat. No. 7,695,427, entitled “Methods and Apparatus for Treating Body Tissue Sphincters and the Like,” issued Apr. 13, 2010, the disclosure of which is incorporated by reference herein, in its entirety; U.S. Pat. No. 8,070,670, entitled “Methods and Devices for Luminal and Sphincter Augmentation,” issued Dec. 6, 2011, the disclosure of which is incorporated by reference herein, in its entirety; and U.S. Pat. No. 8,734,475, entitled “Medical Implant with Floating Magnets,” issued May 27, 2014, the disclosure of which is incorporated by reference herein, in its entirety.

While various kinds and types of instruments have been made and used to treat or otherwise engage anatomical lumens, it is believed that no one prior to the inventors has made or used an invention as described herein.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a cross-sectional side view, taken along a coronal plane of the body, of a biological passage;

FIG. 2 depicts a cross-sectional isometric view, taken along a coronal plane of the body, of a human esophago-gastric junction;

FIG. 3 depicts a top plan view of an example of a sphincter augmentation device;

FIG. 4 depicts a partial, cross-sectional view of a portion of the sphincter augmentation device of FIG. 3;

FIG. 5A depicts a top, cross-sectional view of the sphincter augmentation device of FIG. 3 positioned about an LES, with the sphincter augmentation device in an open and expanded configuration;

FIG. 5B depicts a top, cross-sectional view of the sphincter augmentation device of FIG. 3 positioned about the LES of FIG. 5A, with the sphincter augmentation device in a closed and contracted configuration;

FIG. 6 depicts a perspective view of an example of a sizing instrument that may be used to measure the biological passage of FIG. 1;

FIG. 7 depicts a side elevation view of an inner shaft of the shaft assembly of the instrument of FIG. 6, with an associated actuation assembly;

FIG. 8 depicts a perspective view of the inner shaft of FIG. 7;

FIG. 9 depicts a cross-sectional perspective view of the inner shaft of FIG. 7, taken along line 9-9 of FIG. 8;

FIG. 10 depicts a cross-sectional perspective view of the shaft assembly and end effector of the instrument of FIG. 6;

FIG. 11 depicts a side elevation view of the actuation assembly of FIG. 7;

FIG. 12A depicts a top plan view of the end effector and shaft assembly of FIG. 10 placed adjacent to a lower esophageal sphincter, where the end effector is in a closed and non-contracted configuration;

FIG. 12B depicts a top plan view of the end effector and shaft assembly of FIG. 10 placed adjacent to the lower esophageal sphincter, where the end effector is in an open configuration;

FIG. 12C depicts a top plan view of the end effector and shaft assembly of FIG. 10, where the end effector is in a closed and non-contracted configuration while the end effector surrounds the lower esophageal sphincter;

FIG. 12D depicts a top plan view of the end effector and shaft assembly of FIG. 10, where the end effector is in a closed and contracted configuration while the end effector surrounds the lower esophageal sphincter;

FIG. 13 depicts a perspective view of another example of a sizing instrument that may be used to measure the biological passage of FIG. 1;

FIG. 14 depicts a perspective view of an end effector of the instrument of FIG. 13;

FIG. 15 depicts a perspective view of a grip portion of the instrument of FIG. 13;

FIG. 16 depicts a schematic view of an example of a data processing system that may be used to determine an implant size, including a combination of local and remote data sources;

FIG. 17 depicts a schematic view of another example of a data processing system that may be used to determine an implant size, including a combination of local data sources;

FIG. 18A depicts a perspective view of an endoscopic instrument assembly disposed in an esophagus of a patient, with the esophagus in cross-section, and with a deflectable instrument shaft disposed in the sidewall of the esophagus;

FIG. 18B depicts a perspective view of the endoscopic instrument assembly and esophagus of FIG. 18A, with a sizing instrument extending from the deflectable instrument and extending around the lower esophageal sphincter; and

FIG. 18C depicts a perspective view of the endoscopic instrument assembly and esophagus of FIG. 18A, with a sphincter augmentation device deployed from the deflectable instrument and extending around the lower esophageal sphincter.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

I. Overview of Example of Sphincter Augmentation Device

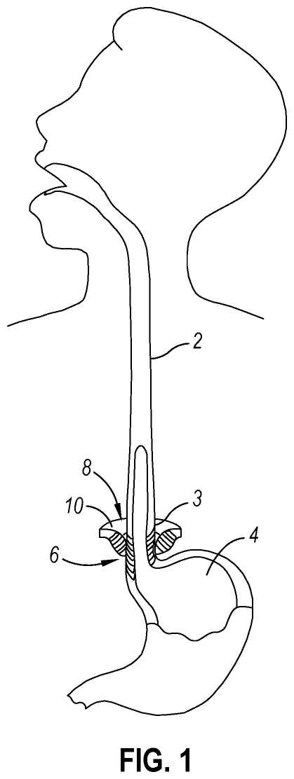

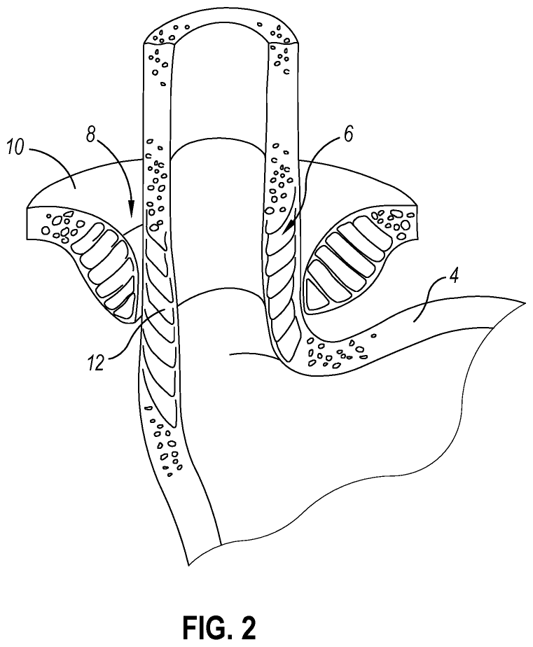

FIGS. 1-2 show selected portions of human anatomy, which includes an esophagus (2) extending from the mouth, through a hiatus (8) defined by a diaphragm (10), and into a stomach (4). Esophagus (2) also includes a distal esophagus (3) and an LES (6). LES (6) is located along distal esophagus (3) adjacent to the junction of esophagus (2) and stomach (4). The portion of LES (6) extending through hiatus (8) is supported by diaphragm (10). When functioning properly, LES (6) is configured to transition between an occluded state and an opened state (as shown in FIG. 2). As best seen in FIG. 2, LES (6) includes a plurality of sling fibers (12). Sling fibers (12) are smooth muscle tissue that may help regulate LES (6) transition between the occluded state and the open state. Hiatus (8) of diaphragm (10) may also help LES (6) transition between the occluded state and the open state.

A healthy LES (6) transitions between the occluded state and the opened state to act as a valve. In other words, a healthy LES (6) may transition from the occluded state to the opened state to allow solids, liquids, and/or gasses to selectively travel between esophagus (2) and stomach (4). For example, a healthy LES (6) may transition from the occluded state to the opened state to permit a bolus of food to travel from esophagus (2) into stomach (4) during peristalsis; or to vent intra-gastric pressure from stomach (4) toward esophagus (2). Additionally, in the occluded state, a healthy LES (6) may prevent digesting food and acidic fluid from exiting stomach (4) back into esophagus (2).

If LES (6) ceases functioning properly by prematurely relaxing, and thereby improperly transitioning esophagus (2) from the occluded state to the opened state, undesirable consequences may occur. Examples of such undesirable consequences may include acidic reflux from stomach (4) into esophagus (2), esophageal damage, inflamed or ulcerated mucosa, hiatal hernias, other GERD symptoms, or other undesirable consequences as will be apparent to one having ordinary skill in the art in view of the teachings herein. Therefore, if an individual has an LES (6) that prematurely relaxes, causing improper transitions from the occluded state to the opened state, it may be desirable to insert an implant around a malfunctioning LES (6) such that the implant and/or LES (6) may properly transition between the occluded state and the opened state.

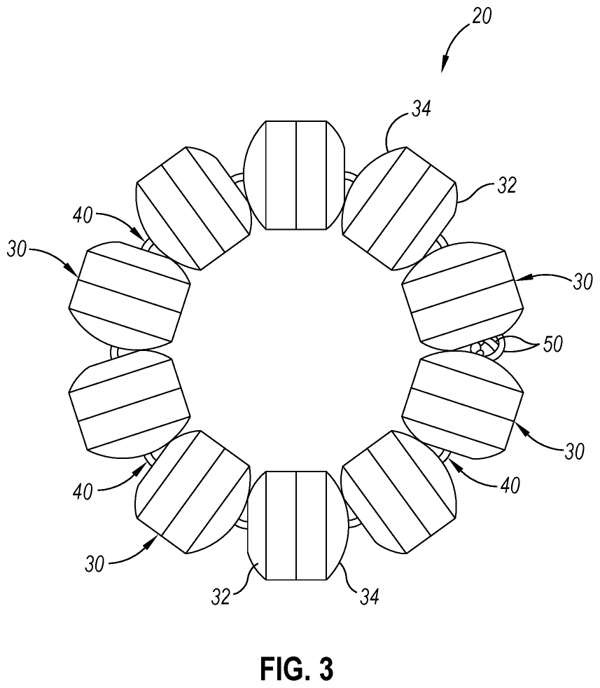

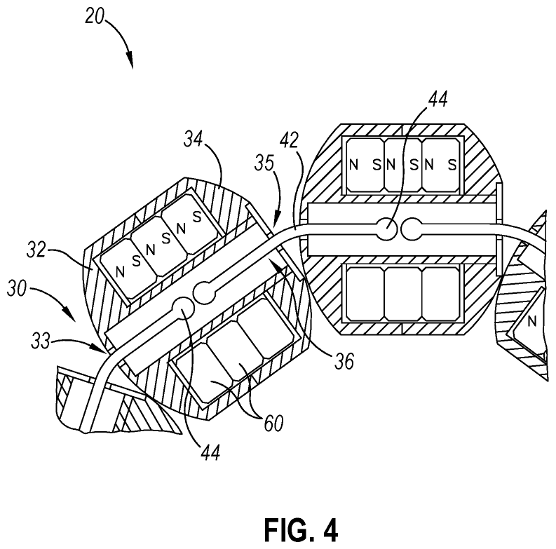

FIGS. 3-5B show an example of a sphincter augmentation device (20) that may be used as an implant around a malfunctioning LES (6) to assist the LES (6) in transitioning between the occluded state and the opened state. Device (20) of this example comprises a plurality of beads (30) that are joined together by a plurality of links (40). Each bead (30) comprises a pair of housings (32, 34) that are securely fastened to each other. By way of example only, housings (32, 34) may be formed of a non-ferrous material (e.g., titanium, plastic, etc.). Each bead (30) further comprises a plurality of annular or toroidal rare-earth permanent magnets (60) that are stacked next to each other within housings (32, 34). In the present example, magnets (60) are completely sealed within beads (30). As best seen in FIG. 4, each bead (30) also defines a chamber (36) that is configured to receive a portion of a respective pair of links (40). Housing (32) defines an opening (33) at one end of chamber (36); while housing (34) defines an opening (35) at the other end of chamber (36).

Each link (40) of the present example comprises a wire (42) that is pre-bent to form an obtuse angle. The free end of each wire (42) terminates in a ball tip (44). Beads (30) are joined together by links (40) such that a first end portion of a link (40) is in one bead (30), a second end portion of the same link (40) is in another bead (30), and an intermediate portion of the same link (40) is positioned between those two beads (30). Chambers (36) of beads (30) are configured to freely receive ball tips (44) and adjacent regions of wires (42); while openings (33, 35) are configured to prevent ball tips (44) from exiting chambers (36). Openings (33, 35) are nevertheless sized to allow wire (42) to slide through openings (33, 35). Thus, links (40) and beads (30) are configured to allow beads (30) to slide along links (40) through a restricted range of motion.

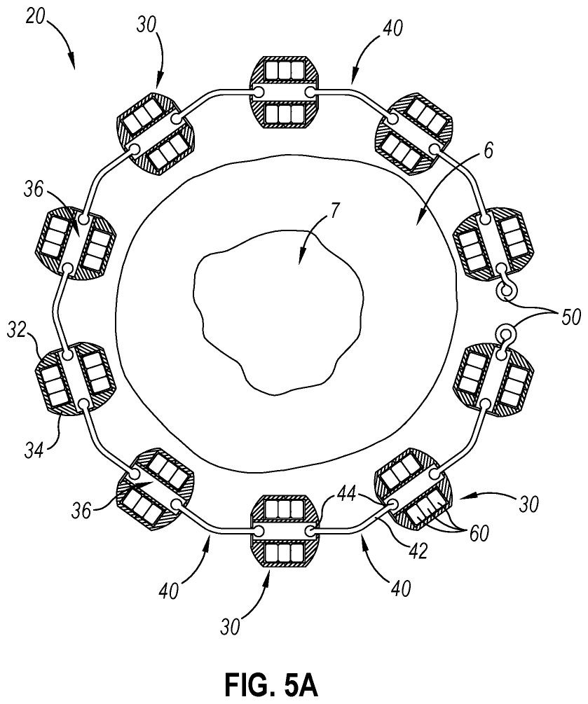

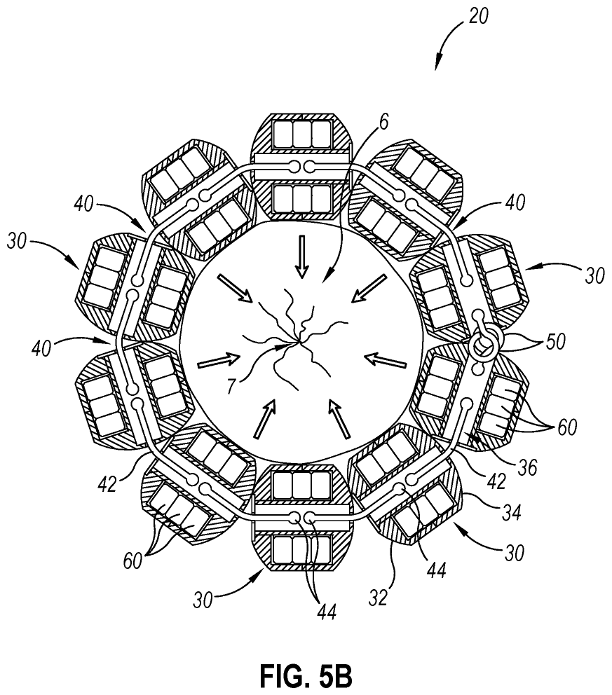

As best seen in FIGS. 5A-5B, two beads (30) have opposing fastener features (50) that allow the ends of device (20) to be coupled together to form a loop. In the present example, fastener features (50) comprise eyelets. In some other versions, fastener features (50) comprise complementary clasp features. As another merely illustrative example, fastener features (50) may be configured and operable in accordance with one or more of the teachings of U.S. Pat. No. 10,405,865, entitled “Method for Assisting a Sphincter,” issued Sep. 10, 2019, the disclosure of which is incorporated by reference herein, in its entirety. Other suitable ways in which the ends of device (20) may be coupled together to form a loop will be apparent to those of ordinary skill in the art in view of the teachings herein. Those of ordinary skill in the art will also recognize that it may be desirable to provide fastener features (50) that can be decoupled if it becomes necessary or otherwise warranted to remove device (20) from the patient.

FIGS. 5A shows device (20) in an open, expanded state, with device (20) being positioned about LES (6). At this stage, the opening (7) defined by LES (6) is in a persistently open state (e.g., allowing the patient to undesirably experience GERD and/or other undesirable conditions), warranting the securement of device (20) about the LES (6). FIG. 5B shows device (20) secured about the LES (6), with device (20) in a closed, contracted state. Device (20) is secured closed via fastener features (50). Magnets (60) are oriented within beads (30) such that each bead (30) will be magnetically attracted to the adjacent bead (30) in device (20). In other words, beads (30) are magnetically attracted to each other to magnetically bias device (20) toward the contracted configuration shown in FIG. 5B.

With device (20) secured around the LES (6) and in the contracted configuration, device (20) deforms the LES (6) radially inwardly to substantially close the opening defined by the LES (6). In doing so, device (20) prevents the patient from experiencing GERD and/or other undesirable conditions that may be associated with a persistently open opening (7) at the LES (6). While magnets (60) have a tesla value that is high enough to substantially maintain opening (7) in a closed state to the point of preventing GERD and/or other undesirable conditions that may be associated with a persistently open opening (7), the tesla value of magnets (60) is low enough to allow LES (6) to expand radially outwardly to accommodate passage of a bolus of food, etc. through the opening (7) of LES (6). To accommodate such expansion, beads (30) may simply slide along links (40) to enlarge the effective diameter of device (20) as the bolus passes. After the bolus passes, the magnetic bias of magnets (60) will return device (20) to the contracted state shown in FIG. 5B. Device (20) thus ultimately prevents GERD and/or other undesirable conditions that may be associated with a persistently open opening (7); while still permitting the normal passage of food, etc. from the esophagus (2) to the stomach (4).

In addition to the foregoing, device (20) may be constructed and operable in accordance with at least some of the teachings of U.S. Pat. No. 7,695,427, the disclosure of which is incorporated by reference herein, in its entirety; and/or U.S. Pat. No. 10,405,865, the disclosure of which is incorporated by reference herein, in its entirety.

II. Example of Sizing Instrument for Anatomical Structure

As mentioned above, certain implants such as device (20) may encompass a malfunctioning LES (6) or another sphincter within the body to suitably assist such sphincters in properly transitioning between the occluded state and the open state. Since the diameter of the LES (6) or another sphincter may vary from patient to patient, it may be necessary or otherwise desirable to vary the length of an implant, to correspond with the diameter of the LES (6) or another sphincter of the patient at hand, to thereby maximize the likelihood of a successful outcome. The suitable length of an implant (e.g., circumference of an implant when attached to the outer diameter of the LES (6) or another sphincter) may be determined by measuring the outer diameter of the LES (6) or another sphincter of the patient at hand. For instance, if an implant includes an array of magnetic elements (e.g., beads (30)), the number of magnetic elements used for a specific implant may be determined by the outer diameter of the LES (6) or another sphincter. The larger the outer diameter, the more magnetic elements will be used; and the smaller the outer diameter, the fewer magnetic elements will be used. In addition to, or in lieu of, varying the number of magnetic elements based on the size of the LES (6), the length of the joining elements (e.g., links (40)) may be varied based on the size of the LES (6).

Since the outer diameter of the LES (6) or another sphincter may vary depending on the patient, and this may influence the configuration of an implant that is to be placed around the LES (6) or another sphincter, it may be desirable to use a sizing instrument having an end effector that is configured to encompass and measure an outer diameter of an LES (6) or another sphincter of an individual patient. An operator may utilize the measurement of the LES (6) or another sphincter to determine what size implant should be used for an individual patient. Upon identifying the appropriate size of the implant, the operator may select the appropriately sized implant from a plurality of available implants. Alternatively, the operator may modify the length of an implant to achieve the appropriate size (e.g., by adding or removing beads (30), selecting an appropriate length of links (40), etc.).

The following describes an example of a sizing instrument (100) that may be utilized to provide a proper engagement between an end effector (170) of sizing instrument (100) and the outer diameter of the LES (6). While sizing instrument (100) is described herein in the context of measuring the LES (6) of the esophagus (2), variations of sizing instrument (100) may be used to measure the outer circumference of any other anatomical passageway, including but not limited to the pylorus, the intestinal region surrounding the ileocecal sphincter, a passageway associated with the sphincter of Oddi, a region of a urethra surrounding the urethral sphincter, a region of the rectum, a region surrounding the upper esophageal sphincter, or any other anatomical passageway.

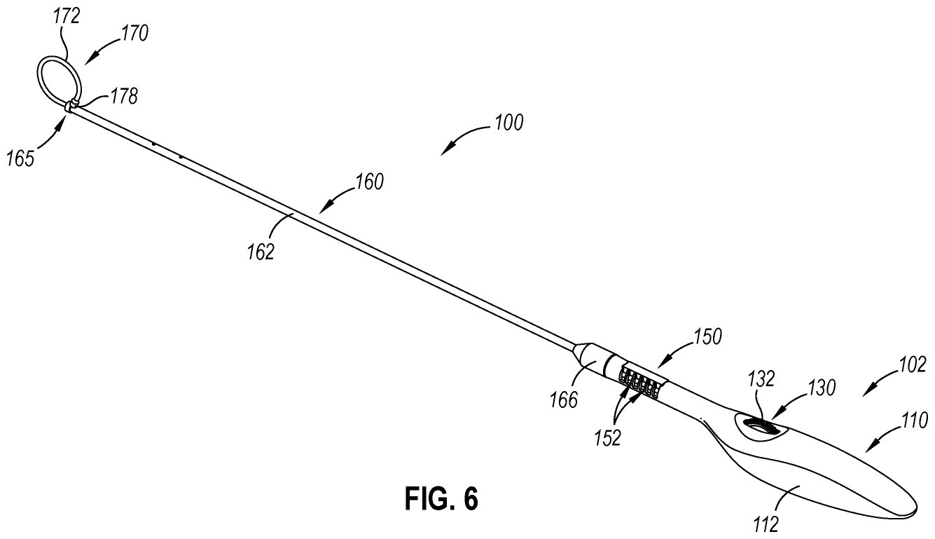

As shown in FIG. 6, sizing instrument (100) of the present example includes a handle assembly (102), a shaft assembly (160) extending distally from handle assembly (102), and an end effector (170) extending distally from shaft assembly (160). Handle assembly (102) includes a grip portion (110), an actuator (130), and a feedback feature (150). Grip portion (110) is formed by a housing (112) and is operable to be grasped by a single hand of an operator. Actuator (130) includes a drive wheel (132) that is positioned to be manipulated by the thumb of the hand that grasps housing (112), such that instrument (100) may be fully operated by just one single hand of the operator. Additional components of actuator (130) will be described in greater detail below. Feedback feature (150) of the present example includes a series of windows (152) formed through housing (112), at the distal end of grip portion (110). Windows (152) are longitudinally spaced apart from each other. As will be described in greater detail below, an indicator (146) of actuator (130) is observable through windows (152) in order to discern the size of an LES (6) that is being measured by instrument (100).

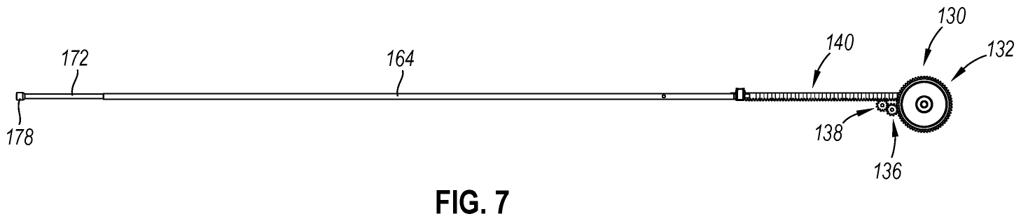

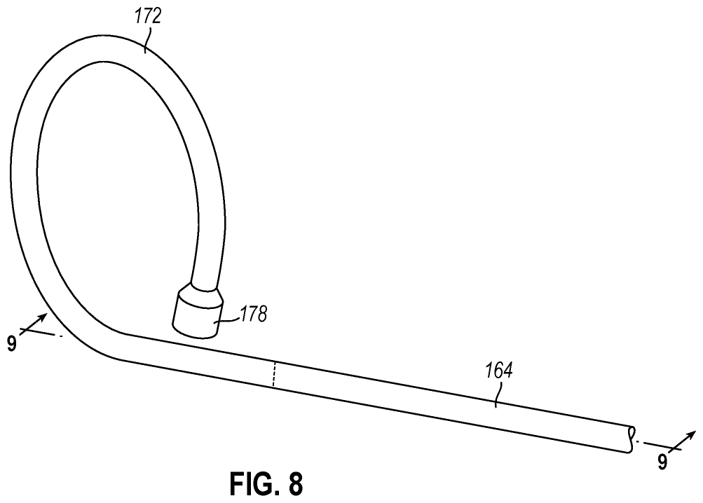

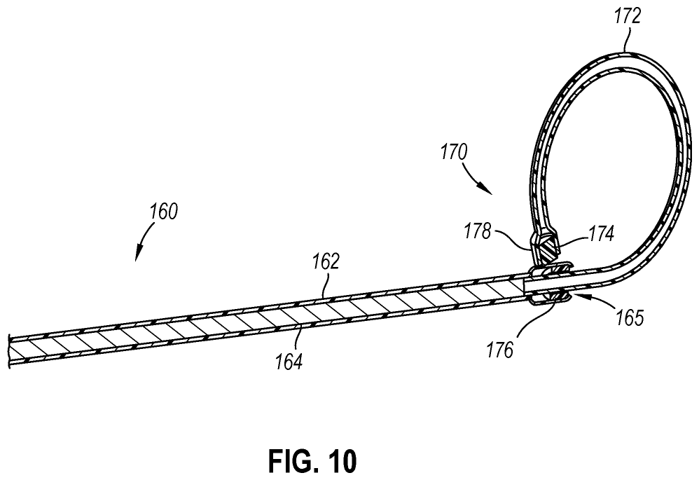

Various components of shaft assembly (160) are shown in FIGS. 6-10. Shaft assembly (160) of this example includes an exterior sheath (162) (FIG. 6) and an inner shaft (164) (FIGS. 7 and 10) slidably housed within exterior sheath (162). Exterior sheath (162) includes an open distal end (165) and a proximal end (166) that is fixedly secured to handle assembly (102). Inner shaft (164) is fixedly secured to a rack (140) of actuator (130) as will be described in greater detail below. In the present example, exterior sheath (162) and inner shaft (164) are both rigid.

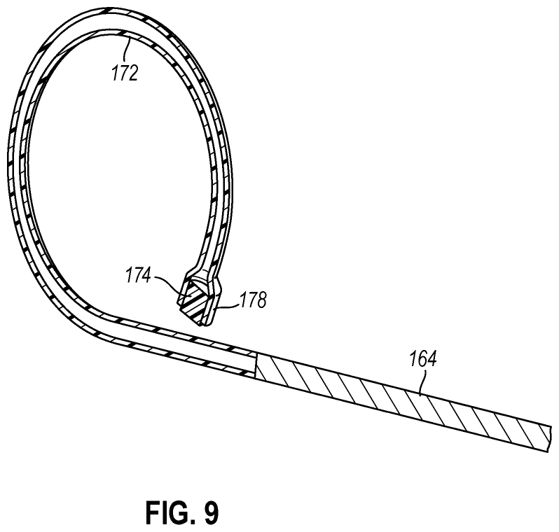

End effector (170) includes a resilient flexible tube (172) extending distally from inner shaft (164), a first magnet (174) (FIGS. 9 and 10) attached to a distal tip (178) of resilient flexible tube (172), and a second magnet (176) (FIG. 10) located at distal end (165) of exterior sheath (162). First and second magnets (174, 176) are attracted to each other such that distal tip (178) of resilient flexible tube (172) is biased toward engagement with open distal end (165) of exterior sheath (162). In some variations, distal end (165) of exterior sheath (162) simply includes a ferrous cuff or other ferrous element that is configured to magnetically couple with first magnet (174); instead of including second magnet (176). Resilient flexible tube (172) defines an adjustable loop and is resiliently biased to assume the loop configuration shown in FIG. 6.

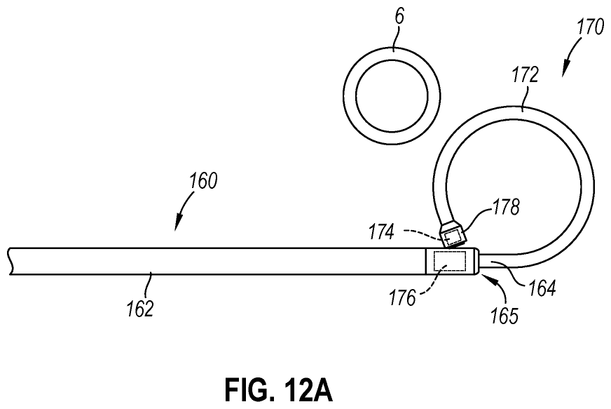

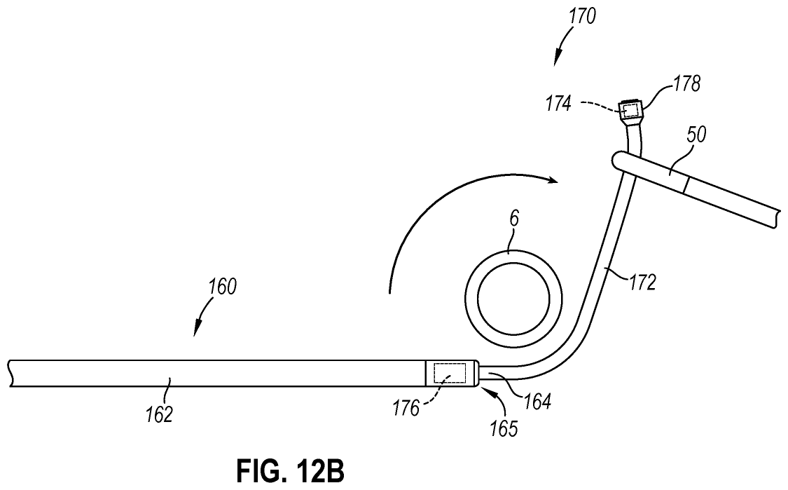

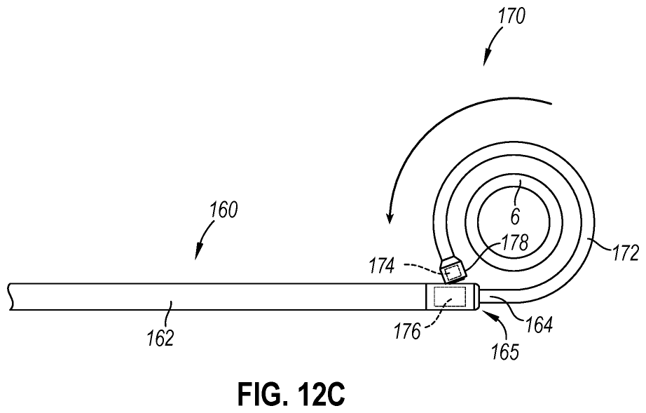

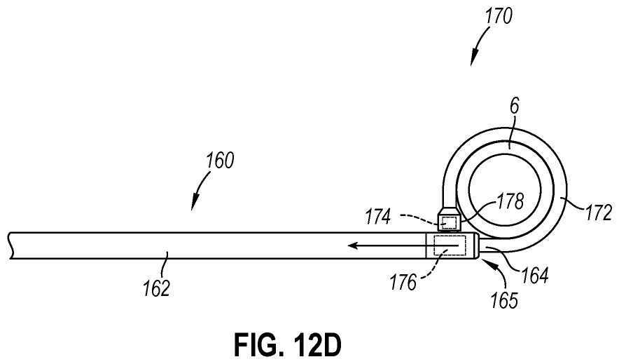

Resilient flexible tube (172) is configured to transition between a closed configuration (e.g., as shown in FIGS. 12A, 12C, and 12D) and an opened configuration (e.g., as shown in FIG. 12B) in order to selectively encompass the LES (6). In the present example, first and second magnets (175, 176) may help ensure that distal tip (178) of resilient flexible tube (172) maintains contact with open distal end (165) of exterior sheath (162) even after the loop defined by resilient flexible tube (172) decreases in diameter as described below. In other words, first and second magnets (174, 176) may help ensure resilient flexible tube (172) remains in a closed configuration as the loop defined by resilient flexible tube (172) decreases in diameter due to the operator's manipulation of actuator (130). In other words, first and second magnets (174, 176) may help ensure resilient flexible tube (172) remains fully encompassed around the LES (6) such that resilient flexible tube (172) may suitably engage the outer diameter of the LES (6) during use of instrument (100).

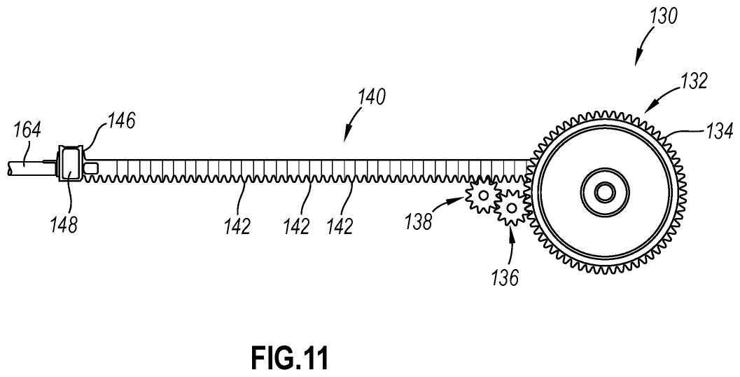

Actuator (130) is operable to decrease the diameter of the adjustable loop defined by resilient flexible tube (172) until resilient flexible tube (172) sufficiently engages the outer diameter of the LES (6) (as shown in FIG. 12D). FIG. 11 shows the components of actuator (130) that are utilized to accomplish the adjustment of the loop defined by resilient tube (172). In this example, actuator (130) includes a drive wheel (132), a pair of idler gears (136, 138), and a rack (140). Drive wheel (132) includes a set of teeth (134) that are exposed relative to housing (112) of handle assembly (102), as best seen in FIG. 6. Thus, when an operator grasps grip portion (110), the operator's thumb may engage teeth (134) and thereby rotate drive wheel (132) relative to housing (112) in order to drive actuator (130). Idler gear (136) meshes with teeth (134) of drive wheel (132), such that rotation of drive wheel (132) rotates idler gear (136). Idler gear (136) meshes with idler gear (138), such that rotation of idler gear (136) rotates idler gear (138). Idler gear (138) meshes with teeth (142) of rack (140), such that rotation of idler gear (138) causes longitudinal translation of rack (140). The proximal end of inner shaft (164) is fixedly secured to the distal end of rack (140), such that translation of rack (140) causes translation of inner shaft (164).

Thus, actuator (130) is operable to convert rotary motion of drive wheel (132) into longitudinal motion of inner shaft (164). As inner shaft (164) translates distally, the diameter of the loop defined by resilient flexible tube (172) increases. As inner shaft (164) translates proximally, the diameter of the loop defined by resilient flexible tube (172) decreases. In other words, as shown between FIGS. 12C-12D, which will be described in greater detail below, movement of resilient flexible tube (172) relative to exterior sheath (162) affects the dimension of the loop defined by resilient flexible tube (172). In particular, the loop defined by tube (172) may become larger in response to distal rotation of drive wheel (132); while the loop defined by tube (172) may become smaller in response to proximal rotation of drive wheel (132).

The size of the loop defined by resilient flexible tube (172) may be determined from the longitudinal position of an indicator (148) on actuator (130) relative to windows (152) and the adjacent markings on handle assembly (102). Indicator (148) is fixedly secured on a flange (146), which is fixedly secured at the distal portion of rack (140). Indicator (148) is positioned to be visible through windows (152) of housing (112) as actuator (130) is operated to translate rack (140) through the operational range of motion of rack (140). The operator may thus visually observe the location of indicator (148) through windows (152) and the adjacent markings to readily ascertain the longitudinal position of rack (140) relative to housing (112). This longitudinal position of rack (140) relative to housing (112) will be further indicative of the diameter of the loop defined by resilient flexible tube (172). This diameter of the loop defined by resilient flexible tube (172) may further indicate the diameter of the LES (6) of the patient at hand, such that the operator may visually observe the location of indicator (148) through windows (152) and the adjacent markings to readily ascertain the diameter of the LES (6) of the patient at hand. This information may enable the operator to determine the size of a device to install around the LES (6) and/or to make any other determination(s) that may be pertinent to the treatment of the patient.

FIGS. 12A-12D show an example of a use of sizing instrument (100). First, as shown in FIG. 12A, an operator may insert end effector (170) and a distal portion of shaft assembly (160) into a patient laparoscopically such that resilient flexible tube (172) is adjacent to the LES (6). During initial insertion of end effector (170) into the patient, flexible tube (172) may be deformed to a straight configuration in order to enable flexible tube (172) to freely pass through a cannula of a trocar or some other passageway into the patient. As shown in FIG. 12A, after end effector (170) has been inserted into the patient, end effector (170) is positioned near the LES (6). Resilient flexible tube (172) is in a closed configuration at this stage. Actuator (130) is in a state where inner shaft (164) is in a distal-most position, such that resilient flexible tube (172) forms the largest loop.

Next, as shown in FIG. 12B, the operator may grasp and pull a portion of resilient flexible tube (172) in order to overcome the biasing force of both tube (172) and magnets (174, 176) to transition resilient flexible tube (172) from the closed configuration to the opened configuration. In the present example, the operator may use a conventional grasping instrument (50) to pull tube (172) into the opened configuration. With resilient flexible tube (172) in the opened configuration, the operator may adjust the position of end effector (170) such that the LES (6) is next to open distal end (165) of exterior sheath (162).

Next, as shown in FIG. 12C, the operator may release resilient flexible tube (172) from grasping instrument (50), such that tube (172) resiliently returns to the closed configuration. Magnetic attraction between magnets (174, 176) may further ensure that distal tip (178) of tube (172) appropriately engages distal end (165) of exterior sheath (162). At this point, the LES (6) is encompassed by resilient flexible tube (172). However, tube (172) is not yet suitably engaged with the LES (6) to the point where feedback feature (150) may be observed to measure the outer diameter of the LES (6). With resilient flexible tube (172) properly encompassing the LES (6) in the closed configuration, the operator may manipulate actuator (130) to retract flexible tube (172) proximally to the point where flexible tube (172) is in full contact with the LES (6) as shown in FIG. 12D. At this point in time, the operator may visually observe the position of indicator (148) relative to windows (152) and the adjacent markings in order to determine the proper size for an implant. After the measurement of the LES (6) attained through observation of feedback feature (150) as described above, the operator may remove instrument (100) from the patient.

It should be understood from the foregoing that, by utilizing instrument (100) as described above, and observing the measured size of the LES via feedback feature (150), the operator may utilize the measurement of the LES (6) to select an implant (e.g., device (20)) that is most appropriate for the patient at hand, to modify an implant (e.g., add or remove beads (30)) so that the implant is at the most appropriate configuration for the patient at hand, and/or for any other purposes.

III. Example of Sizing Instrument with Sensing Features

As described above, it may be beneficial to utilize an instrument like instrument (100) to measure an outer diameter of an LES (6) or another sphincter of an individual patient in order to determine an appropriate size for an implant to install around the LES (6) or another sphincter. While such measurements may be readily obtained via instrument (100), it may be desirable to enhance the functionality of instrument (100) may facilitating access to the LES (6) or another sphincter, such as by modifying shaft assembly (160). It may also be desirable to integrate one or more sensing features into instrument (100) to convert the measurements into digital form. Such digitized measurements may be used to inform the operator of the measurement and/or to communicate with other devices for data processing purposes. The processed data may assist the operator with planning a procedure for installing an implant (e.g., device (20)) around the measured LES (6) or another measured sphincter. An example of a variation of instrument (100) providing such additional functionalities is described in greater detail below.

A. Overview

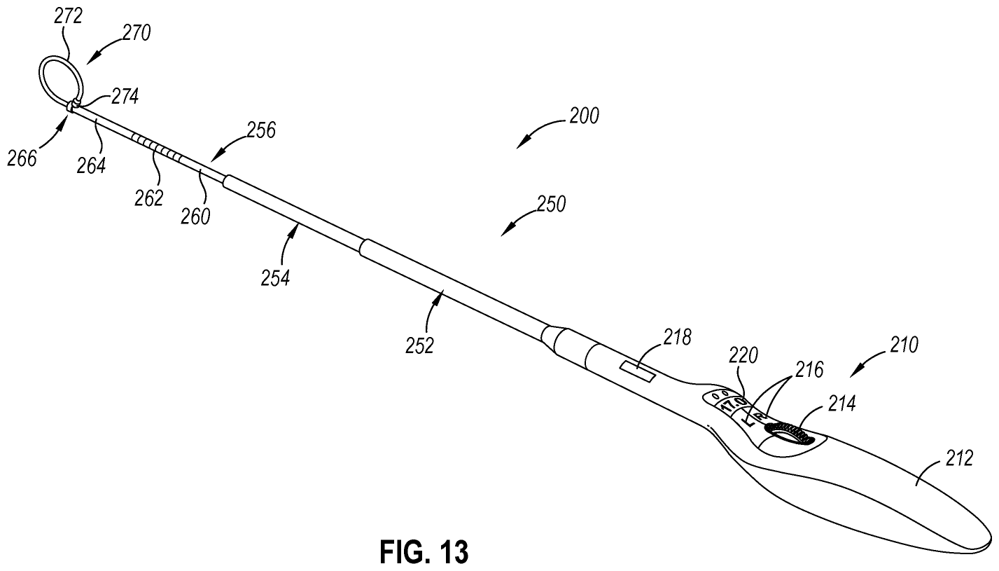

FIG. 13 shows an example of an instrument (200) that is operable to measure an outer diameter of an LES (6) or another sphincter of an individual patient. Instrument (200) of this example may be configured and operable like instrument (100), except as otherwise described below. Instrument (200) includes a grip portion (110), a shaft assembly (250), and an end effector (270). Grip portion (210) includes a housing (212), a drive wheel (214), an articulation control input (216), a telescoping control input (218), and a display (220). Housing (212) is configured for grasping and operation and operation by a single hand of an operator. Drive wheel (214) is operable to drive actuation of end effector (270), much like drive wheel (132) is operable to drive actuation of end effector (170) in instrument (100) as described above. The components linking drive wheel (214) with end effector (270) may be similar to those described above in the context of actuator (130).

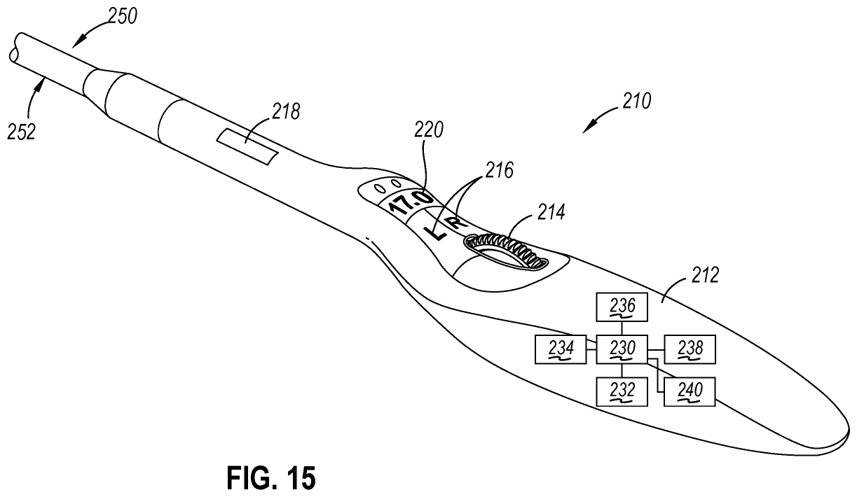

As shown in FIG. 15, grip portion (210) further includes a processing module (230), a battery (232), an inner shaft position sensor (234), an articulation actuator (236), a wireless transceiver (238), and a storage module (240). Battery (232) is operable to provide electrical power to processing module (230), inner shaft position sensor (234), articulation actuator (236), wireless transceiver (238), and any other electrical components that may be powered in instrument (200). While battery (232) is integrated into grip portion (210) in this example, some other variations of instrument (200) may receive power via one or more cables, etc. In some such versions, battery (232) may be omitted. Processing module (230) may execute one or more control algorithms and/or otherwise communicate with inner shaft position sensor (234), articulation actuator (236), wireless transceiver (238), storage module (240) and/or any other electrical components that may be powered in instrument (200). Processing module (230) may include a microprocessor, an ASIC, and/or any other suitable component(s). Examples of how inner shaft position sensor (234), articulation actuator (236), and wireless transceiver (238) may be utilized will be described in greater detail below. Storage module (240) may include a flash memory, an EEPROM, and/or any other suitable form(s) of storage media. In some versions, storage module (240) may store one or more algorithms that may be executed via processing module (230). In addition, or in the alternative, storage module (240) may save data processed via processing module (230), data received via wireless transceiver (238), and/or other data. Storage module (240) may also store data about instrument (200), such as a serial number of instrument (200), a manufacturing lot number of instrument (200), a manufacture date of instrument (200), etc.

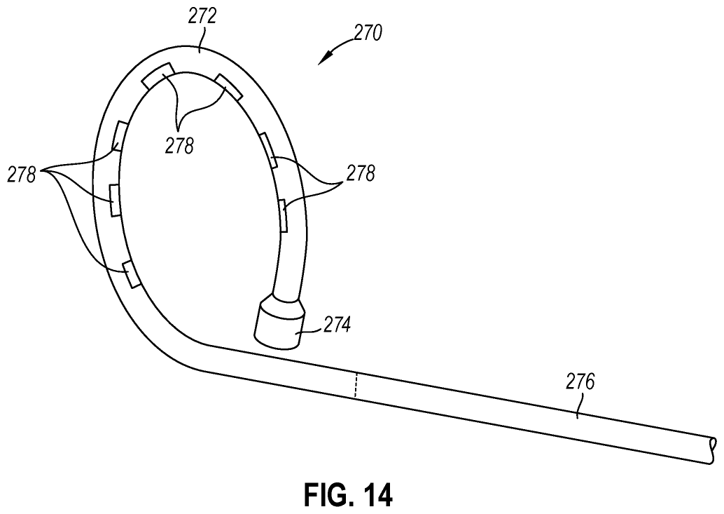

As shown in FIGS. 13-14, shaft assembly (250) of the present example includes a proximal outer shaft portion (252), an intermediate outer shaft portion (254), a distal outer shaft portion (256), and an inner shaft (276). In the present example, outer shaft portions (252, 254) are rigid; while distal outer shaft portion (256) includes an articulation section (262) as will be described in greater detail below. End effector (270) is positioned at the distal end of shaft assembly (250). End effector (270) includes a resilient flexible tube (272) extending distally from inner shaft (276), a first magnet (not shown) attached to a distal tip (274) of resilient flexible tube (272), and a second magnet (not shown) located at distal end (266) of distal outer shaft portion (256). The first and second magnets of end effector (270) are attracted to each other such that distal tip (274) of resilient flexible tube (272) is biased toward engagement with open distal end (266) of distal outer shaft portion (256). Resilient flexible tube (272) defines an adjustable loop and is resiliently biased to assume the loop configuration shown in FIG. 13. End effector (270) of this example is thus configured and operable just like end effector (170) of instrument (100) described above.

B. Example of Telescoping Shaft

In the present example, outer shaft portions (252, 254, 256) are configured to telescope, such that intermediate outer shaft portion (254) may slide longitudinally relative to proximal outer shaft portion (252); and such that distal outer shaft portion (256) may slide longitudinally relative to intermediate outer shaft portion (254). In some versions, proximal outer shaft portion (252) is longitudinally slidable relative to grip portion (210); while in other versions, proximal outer shaft portion (252) is longitudinally fixed relative to grip portion (210). In any case, the telescoping functionality of shaft assembly (250) may allow an operator to change the effective length of shaft assembly (250), which may in turn facilitate access to an LES (6) or another sphincter on a patient-by-patient basis. For instance, the LES (6) of a morbidly obese patient may be positioned at a greater depth from the skin than the LES (6) of a patient who is not morbidly obese, such that it may be beneficial to allow the operator to effectively lengthen shaft assembly (250) in the morbidly obese patient to access the LES (6) of the morbidly obese patient. Conversely, in patients who are not morbidly obese, it may be undesirable to have a shaft assembly (250) that is substantially longer than is necessary to access the LES (6), such that it may provide easier manipulation of instrument (200) to effectively shorten shaft assembly (250) in a patient who is not morbidly obese.

Inner shaft (276) is slidably disposed within outer shaft portions (252, 254, 256).

When an operator adjusts the effective length of shaft assembly (250) by driving relative longitudinal movement of one or more outer shaft portions (252, 254, 256), it may be desirable to drive corresponding longitudinal movement of inner shaft (276) to prevent the loop shape or size of end effector (270) from changing as shaft assembly (250) undergoes the telescoping movement. In some versions, inner shaft (276) includes telescoping shaft portions (not shown) that complement outer shaft portions (252, 254, 256), such that the telescoping shaft portions of inner shaft (276) move in unison with outer shaft portions (252, 254, 256) as shaft assembly (250) undergoes the telescoping movement. In some other versions, the inner shaft (276) lacks telescoping portions, and the entirety of inner shaft (276) moves in unison with outer shaft portions (252, 254, 256) as shaft assembly (250) undergoes the telescoping movement. Alternatively, any other suitable configurations or arrangements may be used to account for telescoping movement of outer shaft portions (252, 254, 256) with respect to inner shaft (276).

As noted above, grip portion (210) includes telescoping control input (218), which may be operated to drive the telescoping movement of shaft assembly (250) as described above. In some versions, telescoping control input (218) comprises a slider, rocker, dial, or other manual user input feature that moves relative to housing (212). A linking mechanism may couple such a form of telescoping control input (218) with shaft assembly (250) to drive the telescoping movement in response to actuation of telescoping control input (218). Thus, some versions of instrument (200) may provide telescoping of shaft assembly (250) that is driven purely manually and mechanically via telescoping control input (218) and an associated linking mechanism. In some other versions, grip portion (210) includes a powered telescoping actuator (not shown) that is powered by battery (232), such that the powered telescoping actuator drives telescoping movement of shaft assembly (250) in response to activation of telescoping control input (218). In some such versions, telescoping control input (218) may include one or more buttons or any other suitable kind(s) of user input feature(s). A powered telescoping actuator may take any suitable form, including but not limited to a solenoid, a motor-driven rack and pinion assembly, a motor-driven screw gear and nut, and/or any other kinds of components.

While shaft assembly (250) of the present example has three outer shaft portions (252, 254, 256), other versions may have more or fewer than three outer shaft portions (252, 254, 256). In some variations, shaft assembly (250) has just one single outer shaft portion. In some such versions, the single outer shaft portion may advance and retract longitudinally relative to grip portion (210) to change the effective length of shaft assembly (250). In some other such versions, the single outer shaft portion may be longitudinally fixed relative to grip portion (210), such that shaft assembly (250) lacks any telescoping functionality at all. It should therefore be understood that the telescoping functionality of shaft assembly (250) described above is merely optional and may be omitted in some versions of instrument (200).

C. Example of Articulation Section

As noted above, distal outer shaft portion (256) of the present example includes an articulation section (262), which is longitudinally interposed between a first shaft segment (260) and a second shaft segment (264) of distal outer shaft portion (256). Articulation section (262) is operable to deflect end effector (270) laterally away from and toward the longitudinal axis defined by shaft assembly (250). Such articulation may facilitate access to an LES (6), such as by enabling the operator to reach around one or more other anatomical structures in order to suitably wrap end effector (270) about the LES (6). In the present example, articulation section (262) is configured to provide lateral deflection of end effector (270) away from and toward the longitudinal axis defined by shaft assembly (250) along one plane, in two directions. In some other versions, articulation section (262) is configured to provide lateral deflection of end effector (270) away from and toward the longitudinal axis defined by shaft assembly (250) along more than one plane.

In order to drive articulation, one or more pull-wires and/or other actuation members may extend along shaft assembly (250). The distal end(s) of the one or more pull-wires and/or other actuation members may be fixedly secured to second shaft segment (264). The proximal end(s) of the one or more pull-wires and/or other actuation members may be joined with articulation actuator (236). In versions where shaft assembly (250) also provides telescoping functionality, the one or more pull-wires and/or other actuation members that drive articulation may translate with telescoping portions of shaft assembly (250) to avoid having the telescoping movement cause lateral deflection at articulation section (262). Alternatively, any other suitable techniques and structural configurations may be used to prevent telescoping movement of shaft assembly (250) cause lateral deflection at articulation section (262).

Articulation actuator (236) may drive longitudinal movement of the one or more pull-wires and/or other actuation members within shaft assembly (250), such that the longitudinal movement of the one or more pull-wires and/or other actuation members within shaft assembly (250) will cause end effector (270) to deflect laterally away from and toward the longitudinal axis defined by shaft assembly (250). In the present example, articulation actuator (236) is powered by battery (232) in response to activation of articulation control input (216). Articulation control input (216) includes a pair of buttons—one button for each direction of lateral deflection relative to the longitudinal axis defined by shaft assembly (250). Alternatively, articulation control input (216) may take any other suitable form. Articulation actuator (236) may also take any suitable form, including but not limited to a motorized spool, a solenoid, a motorized rack and pinion assembly, etc. In some other versions, articulation is driven purely manually, such that neither battery (232) nor any other source of electrical power is used to drive articulation at articulation section (262). Other versions may lack articulation section (262), articulation actuator (236), and articulation control input (216). It should therefore be understood that not all versions of shaft assembly (250) necessarily need to provide articulating functionality.

D. Example of Inner Shaft Position Sensing

As described above in the context of instrument (100), the operator may visually observe the position of indicator (146) of actuator (130) through windows (152) of feedback feature (150) to determine the size of an LES (6) that is being measured by instrument (100), since the position of indicator (146) varies with the position of inner shaft (164). In other words, the longitudinal position of inner shaft (164) is indicative of the size of the LES (6) that is encompassed by end effector (170). With end effector (270) of instrument (200) being configured and operable like end effector (170) of instrument (100), the longitudinal position of inner shaft (276) is also indicative of the size of the LES (6) that is encompassed by end effector (270). However, rather than providing something like indicator (146) that is viewable through windows (152) of feedback feature (150), instrument (200) of the present example provides inner shaft position sensor (234) and display (220).

Inner shaft position sensor (234) is configured to sense the longitudinal position of inner shaft (276) and provide a corresponding signal to processing module (230). In some versions, inner shaft position sensor (234) includes an optical sensor that is positioned and configured to optically track one or more optical markers at a proximal region of inner shaft (276). In some other versions, inner shaft position sensor (234) includes a hall effect sensor that is positioned and configured to optically track one or more magnetic markers at a proximal region of inner shaft (276). In still other versions, inner shaft position sensor (234) effectively senses the longitudinal position of inner shaft (276) by tracking movement of drive wheel (214). For instance, drive wheel (214) may include an integral rotary encoder wheel; and inner shaft position sensor (234) may include an encoder sensor that tracks the rotary encoder wheel of drive wheel (214). Alternatively, inner shaft position sensor (234) may take any other suitable form.

As noted above, inner shaft position sensor (234) may generate signals indicating the longitudinal position of inner shaft (276) and transmit those signals to processing module (230). In some versions, processing module (230) drives display (220) to indicate the size of the patient's LES (6) based on a signal from inner shaft position sensor (234). For instance, display (220) may indicate the outer diameter of the LES (6). Alternatively, display (220) may indicate a size of sphincter augmentation device (20) that is most appropriate for the patient at hand, the number of beads (30) that should be used to form a sphincter augmentation device (20) for the patient at hand, or some other information associated with the size of the patient's LES (6) and/or characteristics of the appropriate sphincter augmentation device (20) for the patient at hand. In the present example, display (220) includes a digital display (e.g., an LED display) that is configured to generate numerical representations. Alternatively, display (220) may take any other suitable form.

In some versions, the data used to formulate the information indicated on display (220) is sourced solely from inner shaft position sensor (234). In some such versions, wireless transceiver (238) is omitted. In some other versions, the information indicated on display (220) is based on a combination of data from inner shaft position sensor (234) and data from one or more other sources, including but not limited to data from remote sources that are in communication with wireless transceiver (238) and/or data from one or more other sensors of instrument (200). Examples of how data from remote sources may be processed in combination with data from inner shaft position sensor (234) are described in greater detail below. Examples of data from other sensors of instrument (200) are also described in greater detail below.

In versions where data from inner shaft position sensor (234) is processed in combination with data from one or more remote sources, such processing may be executed through processing module (230). Alternatively, data from inner shaft position sensor (234) may be communicated to a remote processor via wireless transceiver (238), with the combined data being processed on the remote processor and then further signals being transmitted back to instrument (200) via wireless transceiver (238) for an appropriate rendering of information via display (220). Data from inner shaft position sensor (234) may also be stored on storage module (240). In some versions, data from inner shaft position sensor (234) is initially stored on storage module (240), then later communicated from storage module (240) to a remote processor via wireless transceiver (23 8). In addition to being used to determine the appropriate size of a sphincter augmentation device (20) for the patient at hand and/or other characteristics for a sphincter augmentation device (20) for the patient at hand, data from inner shaft position sensor (234) may be used to develop details of a procedure for implanting a sphincter augmentation device (20) in the patient at hand. Further examples of data processing for developing implantation process details are described in greater detail below.

E. Example of Sensors at End Effector

As shown in FIG. 14, end effector (270) of the present example further includes a plurality of sensors (278). Sensors (278) are positioned along resilient flexible tube (272), proximal to distal tip (274). In some versions, sensors (278) detect whether end effector (270) has fully engaged the tissue of the LES (6). In addition, or in the alternative, sensors (278) may be configured to sense characteristics of tissue of the LES (6) through contact with the tissue. For instance, sensors (278) may sense impedance, capacitance, and/or other characteristics of tissue of the LES (6). Such sensed characteristics may further indicate the density of the tissue of the LES (6) and/or other properties that might influence the selection of a sphincter augmentation device (20) for the LES (6). In in addition, or in the alternative, sensors (278) may be configured to sense characteristics of flexible tube (272) as flexible tube (272) engages the LES (6). For instance, sensors (278) may be configured to sense strain within flexible tube (272). Such strain may further indicate the density of the tissue of the LES (6) and/or other properties that might influence the selection of a sphincter augmentation device (20) for the LES (6). As another example, one or more of sensors (278) may be configured to detect when distal tip (274) of resilient flexible tube (272) is sufficiently engaged with distal end (266) of distal outer shaft portion (256), to thereby indicate when end effector (270) has achieved a closed state around the LES (6) (e.g., similar to what is shown in FIGS. 12C-12D). Display (220) and/or some other kind of user feedback feature may indicate when distal tip (274) of resilient flexible tube (272) is sufficiently engaged with distal end (266) of distal outer shaft portion (256), based on a signal from a corresponding sensor (278).

While a plurality of sensors (278) are shown in FIG. 14, some variations of end effector (270) may include just one single sensor. For instance, sensor (278) may comprise a single wire sensor that extends along at least part of the length of flexible tube (272), with the single wire being positioned to contact the tissue of the LES (6) as end effector (270) is wrapped around the LES (6). In some other variations, sensors (278) are omitted. It should therefore be understood that one or more sensors (278) are not necessarily required in all versions of end effector (270). In versions that include a plurality of sensors (278) different sensors (278) may sense different characteristics. For instance, one or more sensors (278) of end effector (270) may detect impedance of tissue of the LES (6); while one or more other sensors (278) of the same end effector (270) may detect strain in flexible tube (272). It should also be understood that sensors (278) may take a variety of forms. For instance, sensors (278) may comprise electrodes that are positioned to contact tissue of the LES (6). Sensors (278) may also comprise one or more wires embedded within the material forming flexible tube (272).

Sensors (278) communicate signals to processing module (230) in the present example. In some versions, processing module (230) processes data from signals generated by sensors (278) in combination with data from signals generated by inner shaft position sensor (234) and/or in combination with data from one or more remote sources. The combination of this data may be used to generate the information indicated on display (220). In addition, or in the alternative, data from sensors (278) may be communicated to a remote processor and/or remote storage module via wireless transceiver (238). Data from sensors (278) may also be stored on storage module (240). In some versions, data from sensors (278) is initially stored on storage module (240), then later communicated from storage module (240) to a remote processor via wireless transceiver (238). In addition to being used to determine the appropriate size of a sphincter augmentation device (20) for the patient at hand and/or other characteristics for a sphincter augmentation device (20) for the patient at hand, data from sensors (278) may be used to develop details of a procedure for implanting a sphincter augmentation device (20) in the patient at hand. Further examples of data processing for developing implantation process details are described in greater detail below.

While not shown in FIG. 14, some versions of flexible tube (272) may include one or more optical markers and/or one or more radiopaque markers. Such markers may be provided in addition to sensors (278). In some versions, one or more optical markers and/or one or more radiopaque markers are provided on the exterior region of the curve defined by flexible tube (272) while sensors (278) are provided on the interior region of the curve defined by flexible tube (272). Such markers may be useful in the process or wrapping end effector (270) around the LES (6) by promoting visibility via laparoscope and/or other imaging technology. Such markers may also be useful in verifying LES (6) measurement when end effector (270) reaches a closed and contracted configuration around the LES (6) (e.g., similar to what is shown in FIG. 12D). In addition to being positioned on end effector (270), or in lieu of being positioned on end effector (270), one or more optical markers and/or one or more radiopaque markers may be provided in one or more suitable locations along shaft assembly (250). Regardless of whether one or more optical markers and/or one or more radiopaque markers are provided on end effector (270) and/or on shaft assembly (250), it should be understood that instrument (200) may include such marker features to provide interoperability with one or more imaging systems.

F. Example of Data Communication

As noted above, grip portion (210) includes a wireless transceiver (238) in communication with processing module (230). Wireless transceiver (238) is configured to provide wireless communication of data with other devices. In some versions, wireless transceiver (238) is configured to provide wireless communication via Bluetooth or some other wireless modality through which wireless transceiver (238) may be paired with one or more other devices within the same operating room or facility. In some other versions, wireless transceiver (238) is configured to provide wireless communication via WiFi or some other wireless modality through which wireless transceiver (238) may communicate with one or more remote devices (e.g., servers, etc.). In still other versions, wireless transceiver (238) may be omitted. In some such versions, one or more wires may be used to provide communication of data, etc., with processing module (230). Alternatively, processing module (230) may lack the ability to communicate with other devices.

In versions where wireless transceiver (238) is included, communication may be unidirectional or bidirectional. In some unidirectional versions, wireless transceiver (238) may function solely as a transmitter that transmits data from processing module (230) to one or more other devices. In some other unidirectional versions, wireless transceiver (238) may function solely as a receiver that receives data from one or more other devices. In bidirectional versions, wireless transceiver (238) may transmit data from processing module (230) to one or more other devices and receive data from one or more other devices. Examples of information that may be communicated to and/or from instrument (200) via wireless transceiver (238) will be described in greater detail below.

Data that is communicated to and/or from wireless transceiver (238) may be used for various different purposes. By way of example only, data that is communicated to and/or from wireless transceiver (238) may be used to determine an appropriate size of a sphincter augmentation device (20) for the patient at hand and/or to determine other characteristics of an appropriate sphincter augmentation device (20) for the patient at hand. In addition, or in the alternative, data that is communicated to and/or from wireless transceiver (238) may be used to develop details of a procedure for implanting a sphincter augmentation device (20) in the patient at hand. Alternatively, data that is communicated to and/or from wireless transceiver (238) may be used for any other suitable purpose(s).

IV. Example of Data Processing Systems to Determine Implant Size and Guide Implantation Procedure

As described above, a sizing instrument (100, 200) may be used to determine the size of a sphincter in a patient such as the LES (6), and this information may be used to select an implant such as a sphincter augmentation device (20) with an appropriate size and/or other appropriate characteristics for the patient at hand. In some scenarios, it may be beneficial to consider additional information, beyond the measurement of the LES (6), when selecting an implant such as a sphincter augmentation device (20) with an appropriate size and/or other appropriate characteristics for the patient at hand. Such additional information may come from various sources, several examples of which are described in greater detail below.

As noted above, the selection of an appropriate size and/or other characteristics of a sphincter augmentation device (20) may include selecting how many beads (30) are to be included in sphincter augmentation device (20), how long links (40) should be in sphincter augmentation device (20), how strong magnets (60) should be in sphincter augmentation device (20), and/or other variable parameters. In some cases, the selection includes selecting the appropriate sphincter augmentation device (20) from a set of available sphincter augmentation devices (20)—whichever one best fits the parameters identified for the patient at hand—without modifying sphincter augmentation device (20). In some other cases, the selection includes modifying a sphincter augmentation device (20) (e.g., adding or removing one or more beads (30), etc.) to best fit the parameter identified for the patient at hand.

In addition to facilitating the size and/or other appropriate characteristics of an implant such as sphincter augmentation device (20), the data from the LES (6) size measurement and data from other sources may be used to develop ad hoc guidance to a surgeon on how best to install sphincter augmentation device (20), based on characteristics of the patient at hand. Similarly, data from one or more sources may be used to develop ad hoc guidance to a surgeon on how best to insert and operate a sizing instrument (100, 200) for a patient at hand.

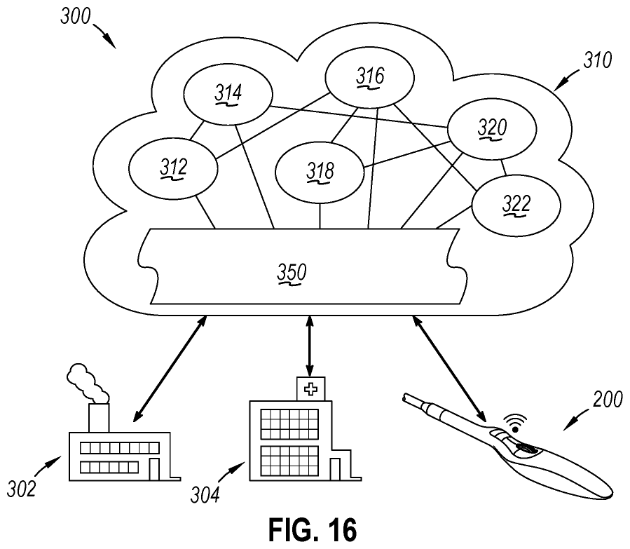

FIG. 16 shows an example of a data processing system (300) that may be used to develop a recommendation of an appropriate size and/or other appropriate characteristics for a sphincter augmentation device (20) based on data from a combination of local and remote sources. As indicated above, data processing system (300) may also be used to develop ad hoc guidance to a surgeon on how best to install sphincter augmentation device (20) and/or other forms of guidance. In this example, data is provided from three separate sources, including the manufacturer (302) of sphincter augmentation device (20), the hospital (304) in which sphincter augmentation device (20) is being installed, and a sizing instrument (200). Data from these sources are combined and processed together, as indicated by cloud (310), on a remote server. For instance, the manufacturer (302) may communicate with cloud (310) via any suitable network connection means; hospital (304) may communicate with cloud (310) via any suitable network connection means; and instrument (200) may communicate with cloud (310) via transceiver (238). In some variations, data communicated from transceiver (238) passes through a portal at hospital (304) to reach could (310).

By way of example only, the data processed in cloud (310) may include patient biometrics (312), patient medical records (314), diagnostic imaging data (316), magnet design specifications (318), a disease state of the patient (320), and the position of the patient (322). Patient biometrics (312) may include data from sensor (234) indicating a measured size of the LES (6), data from sensors (278) indicating one or more characteristics of tissue state at the LES (6), and/or other data from instrument (200). At least some patient biometrics (312) may be collected, and even transmitted, from instrument (200). In addition, patient biometrics (312) may include a height and weight of the patient and/or other kinds of biometric data that are not necessarily captured via instrument (200) but may be transmitted from the hospital (304). Patient medical records (314) may be transmitted from the hospital (304) and may include details from the patient's medical history, details of the patient's current medical status, and/or other information. Diagnostic imaging data (316) may be transmitted from the hospital (304) and may include various kinds of imaging data (e.g., CT scan, MRI scan, ultrasound scan, etc.) of the LES (6) and/or other anatomical structures of the patient. In some such versions, the imaging data shows the inner diameter and the outer diameter of the LES (6). Magnet design specifications (318) may be transmitted from the manufacturer (302) and may include magnet size, field strength, and/or other characteristics of magnets (60) in sphincter augmentation devices (20). Disease state of the patient (320) may be transmitted from the hospital (304) and may indicate the degree to which the patient is suffering from GERD and/or any other condition that may warrant implantation of sphincter augmentation device (20). The position of the patient may be transmitted from the hospital (304) and may indicate whether the patient was supine, in the Trendelenburg position, in the reverse Trendelenburg position, or in some other position at the time an LES (6) measurement was acquired using instrument (200). Of course, various other kinds of data may be processed in cloud (312), in addition to or in lieu of the kinds of data referred to above.

The data may be processed in cloud (312) to determine, among other things, an ad hoc recommendation (350) for the patient at hand. Such a recommendation (350) may include the number of beads (30) to include in a sphincter augmentation device (20) for the patient at hand, the length of links (40) to include in a sphincter augmentation device (20) for the patient at hand, the strength of magnets (60) to include in a sphincter augmentation device (20) for the patient at hand, etc. In addition, or in the alternative, such a recommendation (350) may include suggested details on how the procedure for installing sphincter augmentation device (20) should be carried out for the patient at hand. Such details may include the position(s) for placement of one or more trocars or other access ports in the abdomen of the patient, etc. In some cases, a recommendation (350) may be generated before data is acquired from sizing instrument (200). In some such cases, recommendation (350) includes a recommended position and/or angle for inserting a trocar or other access port to receive sizing instrument (200) in the patient. Such a recommendation (350) may be tailored to provide an orientation of end effector (270) that is perpendicular to the longitudinal axis if the esophagus (2), which may provide a desired fit between end effector (270) and the LES (6). In versions where recommendation (350) is based at least in part on imaging data from a laparoscope, the same laparoscope may be used to visually observe the LES (6) measurement procedure using sizing instrument (200); and/or the sphincter augmentation device (20) installation process.

In some versions of system (300), one or more of the sources (302, 304, 200) of data may also receive data from cloud (312). Such data transmitted from cloud (312) to one or more sources (302, 304, 200) may include recommendation (350). In addition, or in the alternative, data transmitted from cloud (312) to a given one of sources (302, 304, 200) may include data from one or more of the other sources (302, 304, 200). Such bidirectional communication of data from and to sources (302, 304, 200) may promote machine learning and/or other potentially useful data correlations for manufacturer (302), hospital (304), or processing module (230) of instrument (200). While not shown in FIG. 16, data may also be transmitted from cloud (312) to a central repository (not shown) for later access. For instance, manufacturer (302), hospital (304), and/or instrument (200) may access data from the central repository at a later date for quality control purposes, for combining with data associated with another procedure involving another patient, or for any other suitable purpose(s).

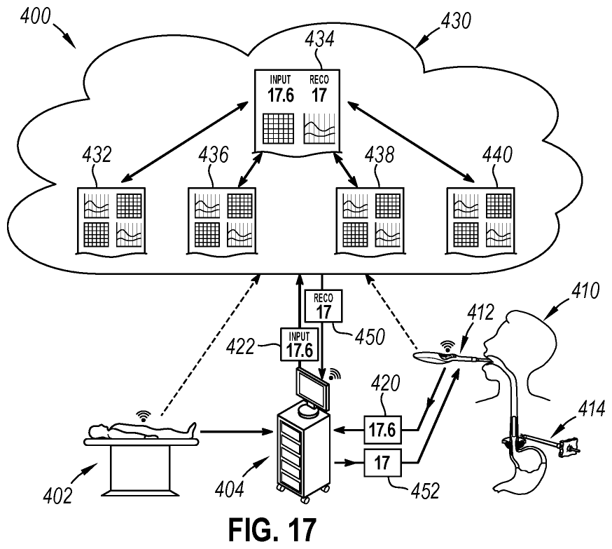

In system (300) shown in FIG. 16, hospital (302) and instrument (200) represent two local sources of data, with hospital (302) and instrument (200) being local relative to the patient. For instance, the patient may be physically located in hospital (302); and instrument (200) may be inserted into the patient. FIG. 17 shows an example of a data processing system (400) that may be used to develop a recommendation of an appropriate size and/or other appropriate characteristics for a sphincter augmentation device (20) based on data from local sources. Data processing system (400) may also be used to develop ad hoc guidance to a surgeon on how best to install sphincter augmentation device (20) and/or other forms of guidance. While remote data sources are not shown in FIG. 17, it should be understood that data processing system (400) may also include data from one or more remote sources. Thus, aspects of system (300) may be combined with aspects of system (400), such that systems (300, 400) should not be viewed as being mutually exclusive of each other.

System (400) of the present example includes a patient data source (402), a console data source (404), and an instrument data source (410). Data from data sources (402, 404, 410) are combined and processed together, as indicated by cloud (430), on a remote server. In some versions, console data source (404) is the only data source directly linked with cloud (430), such that data from patient data source (402) reaches cloud (430) via console data source (404); and such that data from instrument data source (410) reaches cloud (430) via console data source (404). Alternatively, patient data source (402) and/or instrument data source (410) may be directly linked with cloud (430), such that data from patient data source (402) and/or instrument data source (410) does not need to pass through console data source (404) to reach cloud (430).

Patient data source (402) may include various kinds of patient data such as patient identification, medical history (e.g., similar to patient medical records (314) described above), biometric data (e.g., similar to patient biometrics (312) described above), a disease state (e.g., similar to disease state (320) described above), and/or any other suitable kind(s) of information specific to the patient at hand. Console data source (404) may include any of the various kinds of data from hospital (304) described above with reference to FIG. 16 and/or other kinds of data. In some versions, console data source (404) merely serves as a conduit for data between patient data source (402) and cloud (430), and/or between instrument data source (410) and cloud (430), such that console data source (404) does not necessarily provide additional data beyond the data provided from patient data source (402) and/or instrument data source (410).

Instrument data source (410) may provide data from sizing instrument (200), like the data from sizing instrument (200) as described above with reference to FIG. 16. Instrument data source (410) may also provide data from other kinds of instrumentation. For instance, as shown in FIG. 17, instrument data source (410) may provide data from a manometry instrument (412) that is operable to measure pressure or forces within various parts of the gastrointestinal tract of the patient. In some versions, manometry instrument (412) includes one or more sensors, a processing module, and a communication module (e.g., like transceiver (238)) to transmit manometry data directly to cloud (430) and/or to console data source (404). In some other versions, data from manometry instrument (412) is manually entered into console data source (404); then subsequently transmitted from console data source (404) to cloud (430). As also shown in FIG. 17, instrument data source (410) may provide data from a laparoscope (414). In some versions, laparoscope (414) is operable to provide hyperspectral imaging of the LES (6) and/or other anatomical structures. In addition, or in the alternative, laparoscope (414) may be operable to provide structured light imaging of the LES (6) and/or other anatomical structures. In any case, data from laparoscope (414) may provide further information about the LES (6) and/or other anatomical structures of the patient at hand, beyond the information provided by sizing instrument (200) and manometry instrument (412).

By way of example only, the data processed in cloud (430) may include historical data (432), an aggregation of patient medical biometrics (436), an aggregation of sphincter locations (438), and an aggregation of disease conditions (440). This data (432, 436, 438, 440) may be processed in combination with other data from sources (402, 404, 410), as described above, through an algorithm (434). In some variations, at least some of the data (312, 314, 316, 318, 320, 322) described above with reference to FIG. 16, and/or other data, is also processed through algorithm (434). In the present example historical data (432) includes an aggregation of data from follow-up visits from previous procedures. For instance, this historical data (432) may reflect the efficacy of prior recommendations, including whether adjustments to algorithm (434) may be warranted in view of the data from follow-up visits from previous procedures. Historical data (432) may thus enhance machine learning through algorithm (434). Patient medical biometrics (436) may include biometric data associated with the patient at hand; but also an aggregation of biometric data from patients from previous procedures. Patient medical biometrics (436) may thus also enhance machine learning through algorithm (434). Sphincter location data (438) may include data reflecting the positions of the LES (6) of the patient at hand; but also the positions of LESs (6) of patients from previous procedures. Sphincter location data (438) may thus also enhance machine learning through algorithm (434). Disease condition data (440) may include disease condition data associated with the patient at hand; but also an aggregation of disease condition data from patients from previous procedures. Disease condition data (440) may thus also enhance machine learning through algorithm (434). Of course, various other kinds of data may be processed in cloud (430), in addition to or in lieu of the kinds of data referred to above.

The data may be processed in cloud (430) to determine, among other things, an ad hoc recommendation (450) for the patient at hand. Such a recommendation (450) may include the number of beads (30) to include in a sphincter augmentation device (20) for the patient at hand, the length of links (40) to include in a sphincter augmentation device (20) for the patient at hand, the strength of magnets (60) to include in a sphincter augmentation device (20) for the patient at hand, etc. As one illustrative example, in a case where manometry data indicates the patient has a high stomach pressure, recommendation (450) may indicate that relatively strong magnets (60) and/or relatively shorter links (40) should be used to control separation between beads (30). In addition, or in the alternative, a recommendation (450) from algorithm (434) may include suggested details on how the procedure for installing sphincter augmentation device (20) should be carried out for the patient at hand. Such details may include the position(s) for placement of one or more trocars or other access ports in the abdomen of the patient, etc.

In the present example, instrument data source (410) generates a raw data value (420) representing a primary estimate of a suitable ad hoc characteristic of sphincter augmentation device (20) for the patient at hand. For instance, raw data value (420) may indicate that 17.6 beads (30) should be used in a version of sphincter augmentation device (20) for the patient at hand. Since only a whole number of beads may be used, algorithm (434) may be used to refine the raw data value (420) (e.g., to determine whether it should be rounded up to 18 beads (30) or rounded down to 17 beads (30)). To that end, raw data value (420) is first communicated to console data source (404); which then relays raw data value (422) to cloud (430). Once in cloud (430), raw data value (420) is processed in combination with other data (432, 436, 438, 440) through algorithm (434) to yield a refined, recommended data value (450). In this example, the recommended data value (450) indicates that 17 beads (30) should be used in a version of sphincter augmentation device (20) for the patient at hand. This recommended data value (450) is communicated from cloud (430) to console data source (404); which then relays recommended data value (452) to instrument data source (410). The surgeon may then observe recommended data value (452) at instrument data source (410). In some other variations, the surgeon observes recommended data value (452) at console data source (404) (e.g., on a display screen, etc.).

While algorithm (434) of the above-described example provides a recommendation (450) of a recommended size for sphincter augmentation device (20), algorithm (434) may provide other kinds of recommendations (450) in addition to, or in lieu of, providing a recommended size for sphincter augmentation device (20). For instance, algorithm (434) may yield suggested details on how the procedure for installing sphincter augmentation device (20) should be carried out for the patient at hand. Such details may include the position(s) for placement of one or more trocars or other access ports in the abdomen of the patient, etc. In some cases, a version of algorithm (434) may be executed before data is acquired from sizing instrument (200). In some such cases, algorithm (434) may yield a recommended position and/or angle for inserting a trocar or other access port to receive sizing instrument (200) in the patient. Such an output of algorithm (434) may be tailored to provide an orientation of end effector (270) that is perpendicular to the longitudinal axis if the esophagus (20), which may provide a desired fit between end effector (270) and the LES (6). In versions where such an output of algorithm (434) is based at least in part on imaging data from a laparoscope (414), the same laparoscope (414) may be used to visually observe the LES (6) measurement procedure using sizing instrument (200); and/or the sphincter augmentation device (20) installation process.

As described above, a raw data value (420, 422) is communicated to cloud (430) and refined through algorithm (434) to yield a recommendation (450, 452). In the example described above, raw data value (420, 422) includes a proposed number of beads (30) for sphincter augmentation device (20). In some other versions, raw data value (420, 422) may include some other kind of baseline data that is subject to refinement through algorithm (434). For instance, in some versions, one or more of data sources (402, 404, 410) generates a baseline procedure plan starting point. This baseline procedure plan may include various details such as where access ports should be installed in the patient, positions of one or more laparoscopes in the patient, characteristics of sphincter augmentation device (20) to be installed in the patient, the most appropriate positioning for the patient during the sphincter augmentation device (20) installation procedure, preferable instruments to use during the sphincter augmentation device (20) installation procedure, etc. The baseline procedure plan may also include highlights for procedure steps that might be particularly challenging or risky for the patient at hand, based on the collected data. This baseline procedure plan may be processed through algorithm (434) for refinement.

In addition, or in the alternative, algorithm (434) may generate a baseline procedure plan as a recommendation (450); and the surgeon may refine that baseline procedure plan. In some such scenarios, the surgeon may simulate the baseline procedure plan (e.g., using software executed through console data source (404), etc.) and adjustment the procedure plan based on the surgeon's observations from the simulation. Such adjustments may include addition of values, modifications to values, further highlighting procedure steps that might be particularly challenging or risky for the patient at hand, un-highlighting procedure steps that the surgeon does not believe will be particularly challenging or risky for the patient at hand, and/or other kinds of adjustments. After making the adjustments to refine the procedure plan, the surgeon may execute the procedure plan. Regardless of whether the procedure plan is refined through algorithm (434) and/or based on adjustments made by the surgeon, the refined procedure plan may be stored in a library of baseline procedure plans for future reference in subsequent iterations of algorithm (434) for other patients in other procedures. Cloud (430) may thus provide machine learning as described above. Similarly, each time data is processed through algorithm (434) in cloud (430), the resulting recommendation and/or associated data may be further aggregated with the data (432, 436, 438, 440) that was used to execute algorithm (434), such that this resulting recommendation and/or associated data may be utilized in subsequent iterations of algorithms (434) in connection with other patients.

Either or both of systems (300, 400) described above may provide remote storage of any or all of the various kinds of data described above. Such a remote storage system may include a central library database for performing analytics and/or trending evaluation in local demographics. Alternatively, the centralized data may be utilized in any other suitable fashion.

V. Example of Endoscopic Instrument Assembly

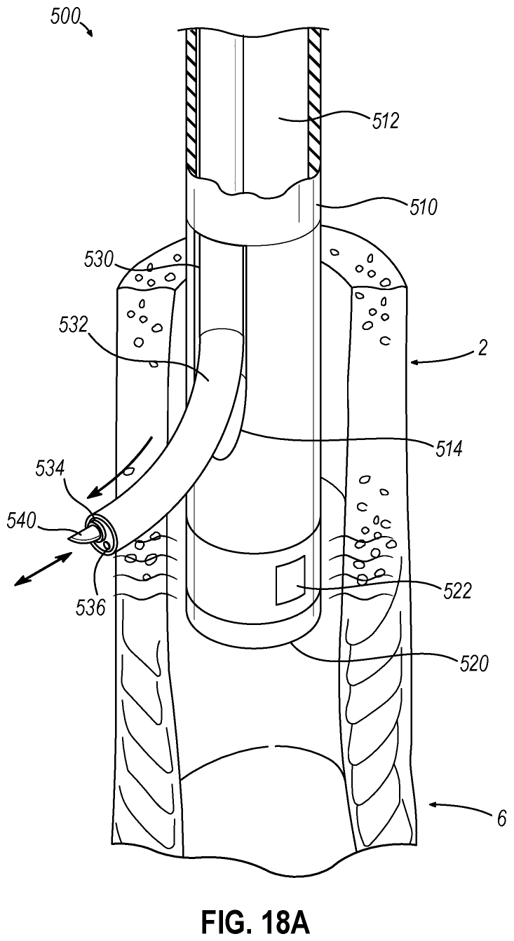

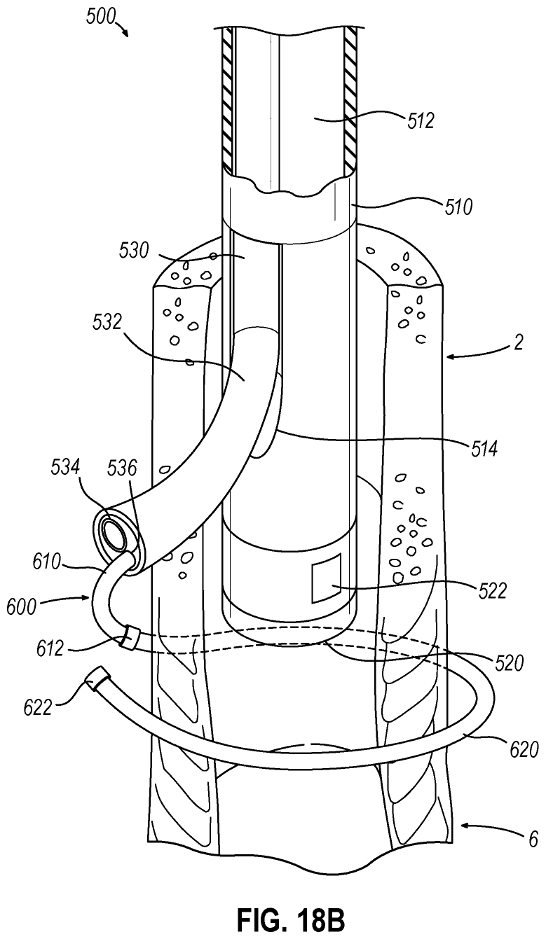

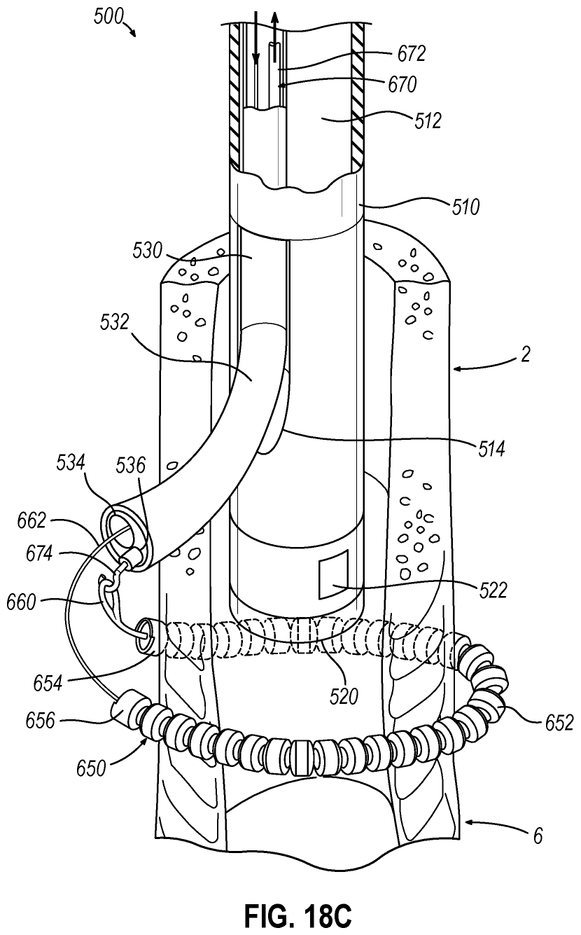

In some procedures, a shaft assembly (160, 250) of a sizing instrument (100, 200) may be introduced into the patient via a trocar or other port inserted through the abdomen of the patient; or through a thoracic incision formed between two ribs of the patient. Similarly, a sphincter augmentation device (20) may be introduced into the patient via a trocar or other port inserted through the abdomen of the patient; or through a thoracic incision formed between two ribs of the patient. In some scenarios, it may be desirable to minimize the number of trocars or other ports in the abdomen of the patient and/or the number of thoracic incisions formed between ribs of the patient, such that it may be desirable to introduce a shaft assembly (160, 250) of a sizing instrument (100, 200) and/or a sphincter augmentation device (20) into the patient through some other passageway. An example of an alternative procedure and associated instrumentation for introducing a sizing instrument and sphincter augmentation device into a patient is shown in FIGS. 18A-18C.

FIG. 18A shows an endoscopic instrument assembly (500) that includes a primary shaft (510) defining an interior (512) and a distal end (520). Primary shaft (510) is sized to be transorally inserted into the esophagus (2), to position distal end (520) at or near the LES (6). An imaging element (522) is positioned proximal to distal end (520) and is configured to provide imaging along a line of sight that is transversely oriented relative to the longitudinal axis of primary shaft (510). Imaging element (522) is configured to assist in the determination of when distal end (520) is positioned at or near the LES (6). In some versions, imaging element (522) is configured to provide hyperspectral imaging, structured light imaging, and/or other kinds of imaging capabilities that may further assist in the determination of when distal end (520) is positioned at or near the LES (6). Some variations of primary shaft (510) may also include a distally-oriented imaging element (not shown) that is configured to provide imaging along a line of sight that is coaxial with, or at least parallel with, the longitudinal axis of primary shaft (510).

Primary shaft (510) also includes a lateral port (514) that is proximal to distal end (520). A deflectable instrument (530) is disposed through lateral port (514) at the stage of operation shown in FIG. 18A. Deflectable instrument (530) includes a steerable distal portion (532), a first distal port (534), and a second distal port (536). Deflectable instrument (530) is configured to slide longitudinally along interior (512) of primary shaft (510), such that deflectable instrument (530) may be in a proximally retracted position relative to lateral port (514) before primary shaft (510) reaches the position shown in FIG. 18A. In such cases, deflectable instrument (530) may be advanced distally relative to primary shaft (510) and thereby exit lateral port (514) after primary shaft (510) reaches the position shown in FIG. 18A. Steerable distal portion (532) is configured to deflect laterally away from the longitudinal axis of primary shaft (510) to thereby achieve the deflected state shown in FIG. 18A. By way of example only, steerable distal portion (532) may be driven by one or more pull wires and/or any other suitable kind of driving actuators. A cutting instrument (540) is configured to advance distally out through first distal port (534). Cutting instrument (540) is sufficiently sharp to penetrate through the sidewall of the esophagus (2). This enables the distal portion of deflectable instrument (530) to pass through the sidewall of the esophagus (2) such that the distal portion of deflectable instrument (530) may be positioned outside the esophagus near the LES (6), as shown in FIG. 18A.

Once the distal portion of deflectable instrument (530) has been positioned outside the esophagus near the LES (6), as shown in FIG. 18A, a sizing instrument (600) may be advanced distally out through second distal port (536), as shown in FIG. 18B. Sizing instrument (600) of this example includes an exterior sheath (610) and an inner shaft (620) slidably disposed in exterior sheath (610). In some versions, exterior sheath (610) is steerable (e.g., via one or more pull-wires, etc.) to achieve a bent configuration as shown in FIG. 18B after the distal portion of exterior sheath (610) exits second distal port (536). In some other versions, exterior sheath (610) is resiliently biased to achieve a bent configuration as shown in FIG. 18B after the distal portion of exterior sheath (610) exits second distal port (536). Alternatively, exterior sheath (610) may be substantially straight upon exiting second distal port (536). Inner shaft (620) may be extended around the LES (620) to thereby encircle the LES (620). In some versions, inner shaft (620) is steerable (e.g., via one or more pull-wires, etc.) to achieve the encircling configuration as shown in FIG. 18B after inner shaft (620) is advanced distally from exterior sheath (610). In some other versions, inner shaft (620) is resiliently biased to achieve the encircling configuration as shown in FIG. 18B after inner shaft (620) is advanced distally from exterior sheath (610). In still other versions, another instrument (e.g., grasping instrument) is used to position inner shaft (620) in the encircling configuration as shown in FIG. 18B after inner shaft (620) is advanced distally from exterior sheath (610).

The distal end of exterior sheath (610) includes a coupling element (612). The distal end of inner shaft (620) also includes a coupling element (622). Coupling elements (612, 622) are configured to join together to complete a loop shape around the LES (6). By way of example only, coupling elements (612, 622) may comprise magnets like magnets (174, 176). In addition, or in the alternative, coupling elements (612, 622) may comprise any other suitable components. Once coupling elements (612, 622) are joined together, inner shaft (620) may be proximally retracted relative to exterior sheath (610) until inner shaft (620) suitably engages the exterior of the LES (6). At this point, the longitudinal position of inner shaft (620) relative to exterior sheath (610) may be checked to determine the outer diameter of the LES (6). In some versions, this is done through visual observation of a mechanical indicator feature (e.g., similar to feedback feature (150) described above). In addition, or in the alternative, this may be done using a sensor (e.g., similar to inner shaft position sensor (234) described above). In any case, the determined outer diameter of the LES (6) may be utilized in any of the various ways described above. While not shown in FIG. 18B, some versions of inner shaft (620) may also include sensors. Such sensors may be configured and operable similar to sensors (278) described above. Similarly, data from such sensors may be utilized in any of the various ways described above.

After the outer diameter of the LES (6) has been determined, endoscopic instrument assembly (500) may be used to install a sphincter augmentation device (650), which is depicted in FIG. 18C. Except as otherwise described below, sphincter augmentation device (650) of the present example may be configured an operable like sphincter augmentation device (20) described above. Sphincter augmentation device (650) of this example includes a plurality of magnetic beads (652) that are joined together by links (not shown), with a pair of clasping features (654, 656) that are configured to join ends of sphincter augmentation device (650) together to complete a loop around the LES (6). A loop feature (660) is positioned at the end with clasping feature (654). A suture (662) extends from the end with clasping feature (656).

Sphincter augmentation device (650) may be introduced into the patient via first distal port (534). Once loop feature (660) is sufficiently exposed relative to first distal port (534), a hook (674) from a manipulator instrument (670) is advanced out through second distal port (536) to engage loop feature (660). Manipulator instrument (670) includes a shaft (672) that is slidably disposed in deflectable instrument (530), with hook (674) being disposed at a distal end of shaft (672). A distal region of shaft (672) may be steerable to wrap the distal region of shaft (672) around the esophagus (2) after the distal region of shaft (6720) exits deflectable instrument (530) via second distal port (536), to thereby reach loop feature (660) with hook (674). After hook (674) engages loop feature (660), manipulator instrument (670) may be proximally retracted relative to deflectable instrument (530) to pull sphincter augmentation device (650) around the LES (6) as shown in FIG. 18C. After sphincter augmentation device (650) substantially encircles the LES (6), loop feature (660) and suture (662) may be manipulated (e.g., using grasping instruments) to join clasping features (654, 656) together.

Once clasping features (654, 656) have been joined together, loop feature (660) and suture (662) may be removed, manipulator instrument (670) may be removed, deflectable instrument (530) may be retracted proximally back into primary shaft (510), and the opening that was formed through the sidewall of the esophagus may be closed (e.g., using sutures, etc.). Endoscopic instrument assembly (500) may then be removed from the patient, leaving sphincter augmentation device (650) installed around the LES (6) of the patient. In some other variations, a separate instrument (e.g., something other than endoscopic instrument assembly (500) is used to assist in the installation of sphincter augmentation device (650) around the LES (6). It should therefore be understood that it is not necessarily required to use the same instrument to introduce sizing instrument (600) (FIG. 18B) and sphincter augmentation device (650) (FIG. 18C) in the patient. Moreover, some variations may provide installation of sphincter augmentation device (650) in the patient in a different procedure (e.g., on a different day) from the procedure in which sizing instrument (600) is used to measure the LES (6).

VI. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus comprising: (a) a body; (b) a shaft assembly extending distally from the body, the shaft assembly including: (i) a first outer shaft member having a first distal end, (ii) a second outer shaft member having a second distal end, the second outer shaft member being translatable relative to the first outer shaft member, a distal portion of the second outer shaft member being configured to encircle an anatomical structure of a patient and couple the second distal end with the first distal end while encircling the anatomical structure of the patient; and (c) a first sensor, the sensor being configured to detect an outer diameter of the anatomical structure encircled by the distal portion of the second outer shaft member.

### Example 2

The apparatus of Example 1, further comprising a display, the display being configured to render a measurement of the outer diameter of the anatomical structure encircled by the distal portion of the second outer shaft member.

### Example 3

The apparatus of Example 2, the display being positioned on the body.

### Example 4

The apparatus of any of Examples 2 through 3, further comprising a processing module, the processing module being in communication with the first sensor, the processing module further being in communication with the display, the processing module being configured to drive the display to render the measurement of the outer diameter of the anatomical structure encircled by the distal portion of the second outer shaft member based at least in part on data from the first sensor.

### Example 5

The apparatus of Example 4, the processing module being positioned in the body.

### Example 6

The apparatus of any of Examples 1 through 5, further comprising a storage module, the storage module being configured to store a measurement of the outer diameter of the anatomical structure encircled by the distal portion of the second outer shaft member.

### Example 7

The apparatus of Example 6, the storage module being positioned in the body.

### Example 8

The apparatus of any of Examples 6 through 7, further comprising a processing module, the processing module being in communication with the first sensor, the processing module further being in communication with the storage module, the processing module being configured to cause the storage module to store the measurement of the outer diameter of the anatomical structure encircled by the distal portion of the second outer shaft member based at least in part on data from the first sensor.

### Example 9

The apparatus of any of Examples 1 through 8, further comprising a data transmission element, the data transmission element being configured to transmit data from the sensor to a data processor outside the body.

### Example 10

The apparatus of Example 9, the data transmission element comprising a wireless transceiver.

### Example 11

The apparatus of any of Examples 9 through 10, the data transmission element being positioned within the body.

### Example 12

The apparatus of any of Examples 1 through 11, further comprising a processing module, the processing module being configured to correlate one or more characteristics of a sphincter augmentation device with the outer diameter of the anatomical structure encircled by the distal portion of the second outer shaft member.

### Example 13

The apparatus of Example 12, the one or more characteristics including a sphincter augmentation device length.

### Example 14

The apparatus of any of Examples 12 through 13, the processing module being configured to correlate one or more characteristics of a sphincter augmentation device with the outer diameter of the anatomical structure encircled by the distal portion of the second outer shaft member based on a combination of data from the first sensor and data from a second source.

### Example 15

The apparatus of Example 14, the second source including preoperative image data.

### Example 16

The apparatus of any of Examples 14 through 15, the distal portion of the second outer shaft member including a second sensor, the second sensor being configured to detect a second characteristic of the anatomical structure encircled by the distal portion of the second outer shaft member, the second sensor providing the second data source.

### Example 17

The apparatus of any of Examples 1 through 16, the shaft assembly including one or more marking elements, the one or more marking elements being visible to an imaging system and configured to indicate positioning of the shaft assembly in the patient.

### Example 18

The apparatus of any of Examples 1 through 17, the shaft assembly being configured to fit through one or both of an esophagus of a human patient or a working channel of a flexible endoscope system.

### Example 19

An apparatus comprising: (a) a body; (b) a shaft assembly extending distally from the body, the shaft assembly including: (i) a first outer shaft member having a first distal end, (ii) a second outer shaft member having a second distal end, the second outer shaft member being translatable relative to the first outer shaft member, a distal portion of the second outer shaft member being configured to encircle an anatomical structure of a patient and couple the second distal end with the first distal end while encircling the anatomical structure of the patient; and (c) a position sensor, the position sensor being configured to detect an outer diameter of the anatomical structure encircled by the distal portion of the second outer shaft member based on a longitudinal position of the second outer shaft member relative to the first outer shaft member.

### Example 20

A method comprising: (a) encircling an anatomical structure of a patient with a shaft member of a sizing instrument; (b) via a sensor of the sizing instrument, determining an outer diameter of anatomical structure; (c) electronically transmitting the determined outer diameter of the anatomical structure to a remote processing system; (d) receiving a sphincter augmentation device recommendation from the remote processing system, the sphincter augmentation device recommendation being based at least in part on the determined outer diameter of the anatomical structure; (e) selecting or modifying a sphincter augmentation device based at least in part on the sphincter augmentation device recommendation; and (f) installing the selected or modified sphincter augmentation device around the anatomical structure of the patient.

VII. Miscellaneous

It should also be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) a body;
(b) a shaft assembly extending distally from the body, the shaft assembly including:
(i) a first outer shaft member having a first distal end,
(ii) a second outer shaft member having a second distal end, the second outer shaft member being translatable relative to the first outer shaft member, a distal portion of the second outer shaft member being configured to encircle an anatomical structure of a patient and couple the second distal end with the first distal end while encircling the anatomical structure of the patient; and
(c) a first sensor, the sensor being configured to detect an outer diameter of the anatomical structure encircled by the distal portion of the second outer shaft member.

2. The apparatus of claim 1, further comprising a display, the display being configured to render a measurement of the outer diameter of the anatomical structure encircled by the distal portion of the second outer shaft member.

3. The apparatus of claim 2, the display being positioned on the body.

4. The apparatus of claim 2, further comprising a processing module, the processing module being in communication with the first sensor, the processing module further being in communication with the display, the processing module being configured to drive the display to render the measurement of the outer diameter of the anatomical structure encircled by the distal portion of the second outer shaft member based at least in part on data from the first sensor.

5. The apparatus of claim 4, the processing module being positioned in the body.

6. The apparatus of claim 1, further comprising a storage module, the storage module being configured to store a measurement of the outer diameter of the anatomical structure encircled by the distal portion of the second outer shaft member.

7. The apparatus of claim 6, the storage module being positioned in the body.

8. The apparatus of claim 6, further comprising a processing module, the processing module being in communication with the first sensor, the processing module further being in communication with the storage module, the processing module being configured to cause the storage module to store the measurement of the outer diameter of the anatomical structure encircled by the distal portion of the second outer shaft member based at least in part on data from the first sensor.

9. The apparatus of claim 1, further comprising a data transmission element, the data transmission element being configured to transmit data from the sensor to a data processor outside the body.

10. The apparatus of claim 9, the data transmission element comprising a wireless transceiver.

11. The apparatus of claim 9, the data transmission element being positioned within the body.

12. The apparatus of claim 1, further comprising a processing module, the processing module being configured to correlate one or more characteristics of a sphincter augmentation device with the outer diameter of the anatomical structure encircled by the distal portion of the second outer shaft member.

13. The apparatus of claim 12, the one or more characteristics including a sphincter augmentation device length.

14. The apparatus of claim 12, the processing module being configured to correlate one or more characteristics of a sphincter augmentation device with the outer diameter of the anatomical structure encircled by the distal portion of the second outer shaft member based on a combination of data from the first sensor and data from a second source.

15. The apparatus of claim 14, the second source including preoperative image data.

16. The apparatus of claim 14, the distal portion of the second outer shaft member including a second sensor, the second sensor being configured to detect a second characteristic of the anatomical structure encircled by the distal portion of the second outer shaft member, the second sensor providing the second data source.

17. The apparatus of claim 1, the shaft assembly including one or more marking elements, the one or more marking elements being visible to an imaging system and configured to indicate positioning of the shaft assembly in the patient.

18. The apparatus of claim 1, the shaft assembly being configured to fit through one or both of an esophagus of a human patient or a working channel of a flexible endoscope system.

19. An apparatus comprising:
(a) a body;
(b) a shaft assembly extending distally from the body, the shaft assembly including:
(i) a first outer shaft member having a first distal end,
(ii) a second outer shaft member having a second distal end, the second outer shaft member being translatable relative to the first outer shaft member, a distal portion of the second outer shaft member being configured to encircle an anatomical structure of a patient and couple the second distal end with the first distal end while encircling the anatomical structure of the patient; and
(c) a position sensor, the position sensor being configured to detect an outer diameter of the anatomical structure encircled by the distal portion of the second outer shaft member based on a longitudinal position of the second outer shaft member relative to the first outer shaft member.

20. A method comprising:
(a) encircling an anatomical structure of a patient with a shaft member of a sizing instrument;
(b) via a sensor of the sizing instrument, determining an outer diameter of anatomical structure;
(c) electronically transmitting the determined outer diameter of the anatomical structure to a remote processing system;
(d) receiving a sphincter augmentation device recommendation from the remote processing system, the sphincter augmentation device recommendation being based at least in part on the determined outer diameter of the anatomical structure;
(e) selecting or modifying a sphincter augmentation device based at least in part on the sphincter augmentation device recommendation; and
(f) installing the selected or modified sphincter augmentation device around the anatomical structure of the patient.
